(19) United States
(12) Patent Application Publication
(10) Pub. No.: US 2015/0202297 A1
(43) Pub. Date: Jul. 23, 2015

(54) DRY POWDER INHALERS COMPRISING A CARRIER OTHER THAN LACTOSE AND A TERNARY COMPONENT

(71) Applicant: Arven Ilac Sanayi Ve Ticaret Anonim Sirketi, Istanbul (TR)

(72) Inventors: Umit Cifter, Istanbul (TR); Ali Turkyilmaz, Istanbul (TR); Onur Mutlu, Istanbul (TR)

(21) Appl. No.: 14/412,066

(22) PCT Filed: Jun. 24, 2013
(86) PCT No.: PCT/TR2013/000192

§ 371 Date: Dec. 30, 2014

(30) Foreign Application Priority Data
Jul. 5, 2012 (TR) 2012/07842
Oct. 2, 2012 (TR) 2012/11213

(51) Int. Cl. A61K 47/26; A61K 9/14; A61K 31/46; A61K 31/439; A61M 15/00; A61K 31/4704; A61K 31/538; A61K 31/27; A61K 31/40; A61K 45/06; A61K 47/12; A61K 31/137

(52) U.S. Cl. CPC A61K 47/26; A61K 47/12; A61K 9/145; A61K 31/46; A61K 31/439; A61K 31/137; A61K 31/4704; A61K 31/538; A61K 31/27; A61K 31/40; A61K 45/06; A61M 15/003; A61M 15/0045; A61M 2202/064

(57) ABSTRACT

This invention relates to novel pharmaceutical compositions for inhalation comprising separately, sequentially or together, drugs having amine in the form of a dry powder in admixture with a pharmaceutically acceptable carrier, other than lactose, and its use in the treatment of respiratory condition selected from asthma and chronic obstructive pulmonary disease (COPD) and other obstructive airways diseases. More particularly, the invention relates to pharmaceutical composition for inhalation further comprising a ternary component. In addition, the present invention relates to novel pharmaceutical composition for inhalation based on combinations of long acting muscarinic antagonists, long acting beta agonists, short acting beta-2 agonists, corticosteroids or a combination of two or more of them.

### FIELD OF THE INVENTION

This invention is a novel pharmaceutical composition for inhalation comprising separately, sequentially or together, drugs having amine in the form of a dry powder in admixture with a pharmaceutically acceptable carrier and its use in the treatment of respiratory condition selected from asthma, chronic obstructive pulmonary disease (COPD) and other obstructive airways diseases. More particularly, the invention relates to pharmaceutical composition for inhalation further comprising a ternary component.

In addition, the present invention relates to novel pharmaceutical composition for inhalation based on combinations of long acting muscarinic antagonists, long acting beta agonists, short acting beta-2 agonists, corticosteroids or a combination of two or more of them.

### BACKGROUND OF THE INVENTION

Amines are organic compounds and functional groups that contain basic nitrogen atom with alone pair. Amines are derivatives of ammonia, wherein one or more hydrogen atoms have been replaced by a substituent such as an alkyl or aryl group. Therefore drugs having amines can be selected from the group comprising aclidinium, glycopyrronium, daratropium, indacaterol, vilanterol, carmeterol, olodaterol or pharmaceutically acceptable salts, or esters thereof, or in enantiomerically pure form or as a racemic mixture.

Most dry powder inhaler (DPI) formulations rely on lactose as a carrier. However, lactose cannot be used for compounds that interact with the reducing sugar function of the lactose. Such drugs are the ones having amine groups, especially having primary or secondary amine.

Another disadvantage can be the Maillard reaction which results from a chemical reaction between an amine group and a reducing sugar. Water content (humidity) and temperature are found to influence the degradation.

Thus, there is a need in the art to look for alternative carriers, other than lactose, that still possess the positive aspects but overcome the above mentioned disadvantages of lactose. Other sugars may comprise but not limited to mannitol, glucose, trehalose, cellobiose, sorbitol and maltitol as potential carriers. Nevertheless, these new carriers may be more sensitive to humidity and extreme temperature according to lactose.

Additionally, carriers are used as a flow aid and facilitate the dose of the active into the lungs. Therefore the properties of the particles of the carrier play an important role in the formulation of dry powder inhaler (DPI). Thus, carriers should be carefully selected, designed and controlled for the use in a dry powder inhalation formulation.

Accordingly, formulations of dry powder Inhalers (DPI) must fulfill a set of requirements, whereby in particular the following are to be considered:

### Content Uniformity of the Active Drug:

In a single dose system, each capsule or blister needs to contain the same amount of drug. In a multi dose system, the same amount of drug must be released every time it is administered, to guarantee that the patient receives the same dose each time. The presence of carrier, such as mannitol, promotes content uniformity in what is generally a low-dosage medication.

### Flowability:

The design of the device, the characteristics of the active and the filling platform to be used will determine the appropriate characteristics of the carrier that will be needed. The flow properties of the formulation will be important to ensure that the overall device functions in the correct way and provides consistent performance. The choice of carrier is essential in ensuring that the device works correctly and delivers the right amount of active to the patient. Therefore to use mannitol as a carrier in two different particle sizes (fine and coarse) is essential.

### Dose Consistency:

DPI devices have to show a consistent dose uniformity in order to guarantee that all doses from the device contain the correct quantity of the active. Regardless of a patient's inhalation ability, it is essential that the dose released by the DPI device is exactly the same every time. Therefore, using mannitol as a carrier with the right properties in the formulation assists dose-consistent delivery.

Accordingly, in order to penetrate into the deep lungs the active particles will have a particle size less than 10 microns and often lower than 4 microns. These small drug particles will have a tendency to agglomerate. By using the appropriate carrier (such as mannitol) this drug to drug agglomeration can be prevented. Furthermore, the carrier (such as mannitol) will help to control the flowability, the drug release from the device and helps to ensure the correct and consistent dosage of the active that reaches the lungs.

Additionally, the formulation should be a homogeneous mixture where the drug particles adhere to the carrier. The adhesion should not be too strong as the drug will not be able to release from the carrier particle during inhalation. Furthermore, a low dose of powder should be filled into the device and the drug should always be released in the same way. One of the main important parameters for the formulation is the particle size of the carrier. Therefore, it is found that using the right ratio of the fine (small) and coarse (large) particles of the selected carrier in the present formulations of the invention is essential.

To fulfill all these requirements the formulations of DPIs needs to be adapted in particular by a careful selection of the carriers used. In order to meet these requirements, the inhalable, fine or microfine particles of active compounds are mixed with carriers. By means of the mixing process, the particle size of the carrier can also be changed such that a certain proportion is inhalable. The particle size of the carrier employed depends on the requirements and specifications of the powder inhaler which is intended for the administration of the formulation. It is true for these mixtures that during all required processing, transport, storage and dosage operations no segregation must take place, i.e. the active compound particles must not detach from their carrier particles. During dispersion in the inhaler, induced by the respiratory flow of the patient, the active compound particles, however, must be detached as effectively as possible, i.e. as quantitatively as possible, in order to be inhaled.

In prior art, there are several compounds used for the treatment of asthma but all these compounds include lactose as a carrier. None of them comprise mannitol, glucose, trehalose, cellobiose, sorbitol or maltitol as potential carriers may be because of their sensitivity to humidity and extreme temperature. This problem is also solved by using additional ternary components.

Ternary compounds can be magnesium stearate, stearic acid, sodium lauryl sulphate, sodium stearyl fumarate, stearyl alcohol and sodium benzoate, could turn out to be suitable depending on the type of carrier and drug used. Especially the preferred one is magnesium stearate.

In prior art there are several examples of magnesium stearate use in inhalation formulations and mostly they suggest to coat the carrier with magnesium stearate or additionally to use it with other additives. Although the respirable fraction increased when magnesium stearate was added, the known amount (min. 1.5% up to 5%) is too much and reduces the mechanical stability of the mixture before use. Furthermore, magnesium stearate is poorly water soluble, its presence in such amount may rise some concerns as to a potential irritation or toxicity of this excipient, part of which can be inhaled by the patient together with the active ingredient. According to prior art, these disadvantages can be solved by adding other additives such as silicon dioxide (aerosil), amino acids (leucine, lysine, valine, methionine or phenylalanine). Mostly L-leucine is preferred. As it is known that inhalable powders are so sensitive such that they have to be used in min. amounts and no other further additives may preferred because of their concern to toxicity.

Finally we have found that the introduction of magnesium stearate in such a small amount is safe and does not produce any toxicologically relevant effect after repeated administration.

Thus, there is still a need for the dry powder inhalation formulations comprising carriers other than lactose that are able to overcome the problems mentioned above and the problems related with interaction of carrier between the drugs having amine and furthermore the problems related to pulmonary administration of drugs and additionally able to overcome the stability problems with the use a ternary compound. This invention also proposes the possibility to obtain different compositions and composition of combinations for pulmonary administration having satisfactory properties in terms of increasing drug deposition or accelerating drug release rate in a safe and effective way.

### THE DETAILED DESCRIPTION OF THE INVENTION

This invention relates to novel pharmaceutical compositions for inhalation comprising separately, sequentially or together, drugs having amine in the form of dry powder in admixture with a pharmaceutically acceptable carrier, other than lactose which doesn't interact with the drugs especially having an amine group and further comprising a ternary component. The present invention further relates its use in the treatment of respiratory condition selected from asthma and chronic obstructive pulmonary disease (COPD) and other obstructive airways diseases.

Accordingly, the main object of the present invention is to provide pharmaceutical compositions for inhalation which is stable throughout the shelflife, in other words, which prevents any chemical reaction between an amine group and reducing sugar which may cause degredation of the active and furthermore resistant to humidity and extreme temperature which may occur during the manufacturing process.

Ternary compounds which are suitable for this invention can be magnesium stearate, stearic acid, sodium lauryl sulphate, sodium stearyl fumarate, stearyl alcohol and sodium benzoate or their mixtures, could turn out to be suitable depending on the type of carrier and drug used. Especially the preferred one is magnesium stearate. The preferred magnesium stearate can be in crystalline or amorphous form. Therefore, magnesium stearate is used to protect the drug, from moisture for inhalation use for stability purposes.

It has surprisingly been found that magnesium stearate is able to minimize the influence of penetrating moisture during the storage of the inhalation powder and stabilize the dry powder formulation. Thus, the quality of the pharmaceutical formulation remains considerably better than conventional formulations which are free of magnesium stearate even on storage under extreme conditions of temperature and humidity.

In addition to general moisture protection, it is found that magnesium stearate also stabilizes the carrier materials and active compounds by suppressing or slowing down undesirable morphological phase transitions.

It has also been found that magnesium stearate is suitable for improving the moisture resistance of any desired dry powder formulations.

Finally we have found that the introduction of magnesium stearate in such a small amount is safe and does not produce any toxicologically relevant effect after repeated administration.

Therefore, the amount of magnesium stearate can be, 0.05 to 2.0% by weight, in particular 0.1 to 1.0% by weight, more particularly it is 0.15 to 0.5% by weight, based on the total formulation.

The particle size may also important and therefore the preferred particle size of magnesium stearate is d10: 0.25-2.5, d50: 3.0-7.0, d90: 7.0-20.0.

The use according to the invention of magnesium stearate is furthermore especially advantageous for use in multidose dry powder inhalers which contain a powder reservoir from which the individual doses are withdrawn by means of a dosage mechanism. The use of magnesium stearate, however, is also suitable for improving the resistance to moisture of predosed units, which can be present, for example, in the form of capsules.

Another main object of the present invention is to provide pharmaceutical compositions for inhalation having an adequate content uniformity of the active in order to guarantee that the patient receives the same dose each time even in low-dosage formulations.

Another object of the present invention is to obtain the dose consistency of the active in order to guarantee that all doses from the device contain the correct quantity of the active. Using the carrier with the right properties and the right ratio in the formulation assists dose-consistent delivery. According to this preferred embodiment, the weight ratio of the fine carrier particles to coarse carrier particles, is between 0.01-0.25 by weight, preferably it is between 0.05-0.20 by weight.

According to a further embodiment of the invention, the fine carrier particles of the said pharmaceutically acceptable carrier have a particle diameter of d₁₀ between 1.0-4.0 μm, d₅₀ between 4.0-7.0 μm and d₉₀ between 7.0-15.0 μm. The coarse carrier particles of the said pharmaceutically acceptable carrier have a particle diameter of d₁₀ between 10.0-50.0 μm, d₅₀ between 50.0-75.0 μm and d₉₀ between 75.0-250.0 μm.

The coarse carrier particles are used to prevent (re)agglomeration of the fine particles of active. To provide this effect, carrier with a particle size of approximately ten times that of the active is used. Generally, a monolayer of the active particles is formed on the larger carrier particles. Since the active and carrier will have to be separated during inhalation, the shape and the surface roughness of the carrier particles is of significant importance. Carrier particles with a smooth surface will separate from the active more easily than highly porous particles of equal size.

The fine carrier particles are used to help the active to reach the lungs in a safer way and higher doses. Because the surface energy is normally not equally spread over the carrier particle, the active will tend to concentrate on higher energy sites. This can make separation of the active from the carrier following pulmonary delivery more difficult, especially for low dose formulations. The presence of fine carrier particles, smaller than 10.0 micron or 5.0 micron, will help to prevent this, as the high energy sites will be occupied by the fine carrier particles and the active will tend to attach to the low energy sites. It is found that lung deposition will increase with an increasing fraction of fine carrier particles. Accordingly a reduction in particle size (having finer particles) increases the fluidization energy and this enhances the increase of the amount of drug particles that will get into the lung.

The drug particles will then adhere to the lower adhesion sites and will be easier released during inhalation. With the addition of fines also the surface area increases significantly and the payload will be reduced. When the fine carrier particles are slightly coarser then the drug particles it could eliminate the friction forces between drug and carrier/mannitol in the mixing process.

Another object of the present invention is to obtain good flowability of the formulations to ensure that the right amount of the active is delivered by the devices for DPIs. In other words, in order to guarantee consistent production of the formulations, mechanical filling of the powder inhaler, correct dosage and release by the powder inhaler the present invention provides free-flowing formulations by selecting the right carrier.

Another object of the present invention is to prevent agglomeration by using appropriate carrier, other than lactose. Active particles have fine or sometimes microfine particles in order to penetrate into the deep lungs. Thus, these small drug particles will have a tendency to agglomerate.

In a preferred embodiment according to the present invention, the pharmaceutically acceptable carrier, other than lactose, is selected from the group comprising mannitol, glucose, trehalose, cellobiose, sorbitol, maltitol or a combination of two or more of them.

As a further embodiment, the carrier may be a combination of mannitol and glucose, or mannitol and trehalose, or mannitol and sorbitol, or mannitol and cellobiose, or mannitol and maltitol.

In a more preferred embodiment, the invention suggests to use mannitol as a carrier, more specifically to use spray-dried mannitol to achieve the best results.

In an ideal drug-carrier system, the adhesion of the active to the carriers is strong enough to prevent demixing during filling, handling and storage, but not so strong, since the active and carrier will have to be separated during inhalation. Therefore the shape and the surface roughness of the carrier particles is of significant importance. It is found that spray-dried mannitol particles will separate from the active more easily than highly porous particles of equal size. Because spray dried mannitol produces more spherical particles and a smooth surface. Such particles are characterized by a lower area of contact and a smaller, more homogeneous particle size distribution that result in a higher respirable fraction than mechanically micronized carriers. One of the advantages for using spray-dried mannitol is to achieve particle diameters of several micrometers with a narrow particle size distribution. This ensures, assuming an appropriate diameter, a maximum deposition of the embedded drug in the tracheo-bronchial and deep alveoli regions at normal inhalation rates. Moreover, spray-dried mannitol exhibited a narrow particle size distribution which means the ratio between the median particle size (d₅₀) and d₉₀ which is equal to or greater than 0.40. Preferably, the ratio between the median particle size and d d₉₀ is between 0.45 and 0.50, more preferably it is 0.50 and 0.70.

Additionally, this narrow particle size distribution also applies to mannitol in the compositions of the present invention which is equal to or greater than 0.40. Preferably, the ratio of narrow particle size distribution is between 0.45 and 0.50, more preferably it is 0.50 and 0.70.

According to a preferred embodiment of the present invention, the average particle diameter (d₅₀) of the drugs having amine is between 1.5-2.5 μm, and the amine is primary amine and/or secondary amine.

According to a preferred embodiment of the present invention, the drug having amine is aclidinium or a pharmaceutically acceptable salt or ester thereof, or in enantiomerically pure form or as a racemic mixture. Preferably it can be aclidinium bromide.

According to a preferred embodiment of the present invention, the drug having amine is glycopyrronium or a pharmaceutically acceptable salt or ester thereof, or in enantiomerically pure form or as a racemic mixture. Preferably it can be glycopyrronium bromide or glycopyrronium acetate.

According to a preferred embodiment of the present invention, the drug having amine is darotropium or a pharmaceutically acceptable salt or ester thereof, or in enantiomerically pure form or as a racemic mixture. Preferably it can be darotropium bromide.

According to a preferred embodiment of the present invention, the drug having amine is indacaterol or a pharmaceutically acceptable salt or ester thereof, or in enantiomerically pure form or as a racemic mixture. Preferably it can be indacaterol maleate.

According to a preferred embodiment of the present invention, the drug having amine is vilanterol or a pharmaceutically acceptable salt or ester thereof, or in enantiomerically pure form or as a racemic mixture. Preferably it can be vilanterol trifenatate.

According to a preferred embodiment of the present invention, the drug having amine is carmoterol or a pharmaceutically acceptable salt or ester thereof, or in enantiomerically pure form or as a racemic mixture. Preferably it can be carmoterol hydrochloride.

According to a preferred embodiment of the present invention, the drug having amine is olodaterol or a pharmaceutically acceptable salt or ester thereof, or in enantiomerically pure form or as a racemic mixture. Preferably it can be olodaterol hydrochloride.

Asthma, chronic obstructive pulmonary disease and other related disorders have been known to be treated with beta-2 adrenergic receptor agonists as they provide a bronchodilator effect to the patients, resulting in relief from the symptoms of breathlessness. Beta-2 adrenergic receptor agonists can be short acting for immediate relief, or long acting for long-term prevention, of asthma symptoms. Long actings are long acting beta agonists (LABA) whose effect lasts for 12 hours or more. In a preferred embodiment, LABAs are selected from the group comprising salmeterol, formoterol, arformoterol, indacaterol, olodaterol, vilanterol, carmoterol, bambuterol or a pharmaceutically acceptable salt or ester thereof, or in enantiomerically pure form or as a racemic mixture or a combination of two or more of them. Preferably LABAs can be salmeterol xinofoate, arformoterol tartarate, indacaterol tartarate, olodaterol hydrochloride, vilanterol trifenatate, carmoterol hydrochloride, bambuterol hydrochloride, formoterol fumarate or a combination of two or more of them.

Short actings are short acting beta-2 agonists (SABA). They are bronchodilators. They relax the muscles lining the airways that carry air to the lungs within 5 minutes, increasing airflow and making it easier to breathe. They relieve asthma symptoms for 3 to 6 hours. They do not control the inflammation. In a preferred embodiment, SABAs are selected from the group comprising salbutamol, levosalbutamol, terbutaline, pirbuterol, procaterol, fenoterol, biltolterol, ritodrine, metaproterenol or a pharmaceutically acceptable salt or ester thereof, or in enantiomerically pure form or as a racemic mixture or a combination of two or more of them. Preferably SABAs can be salbutamol sulphate, salbutamol hemi-sulphate, levosalbutamol sulphate, terbutaline sulfate, pirbuterol hydrochloride, pirbuterol acetate, procaterol hydrochloride, fenoterol hydrobromide, bitolterol mesylate, ritodrine hydrochloride, metaproterenol sulfate or a combination of two or more of them.

Whilst it is also known that, beta-2 agonists provide symptomatic relief of bronchoconstriction in patients, another component of asthma, i. e. inflammation, often requires separate treatment. According to this, involves treatment with a steroid. Treatment with an inhaled corticosteroid is considered one of the most potent and effective therapies currently available for persistent asthma. In a preferred embodiment, inhaled corticosteroids are selected from the group comprising fluticasone, ciclesonide, budesonide, mometasone, beclomethasone, triamcinolone, flunisolide, dexamethasone or a pharmaceutically acceptable salt or ester thereof, or in enantiomerically pure form or as a racemic mixture or a combination of two or more of them. Preferably, the corticosteroids can be fluticasone propionate, fluticasone furoate, ciclesonide, budesonide, mometasone furoate, beclomethasone dipropionate, triamcinolone acetonide, flunisolide acetate, dexamethasone sodium phosphate or a combination of two or more of them.

Bronchoconstriction and inflammation are also associated with bronchial plugging with secretions, which may be treated with long acting muscarinic antagonists (LAMA). In a preferred embodiment LAMAs are selected from the group comprising tiotropium, glycopyrronium, ipratropium, aclidinium, oxitropium or a pharmaceutically acceptable salt or ester thereof, or in enantiomerically pure form or as a racemic mixture or a combination of two or more of them. Preferably the LAMAs can be tiotropium bromide, glycopyrronium bromide, glycopyrronium acetate, ipratropium bromide, aclidinium bromide, oxitropium bromide or a combination of two or more of them.

According to this preferred embodiment of the present invention, the said pharmaceutical compositions may further comprise one or more additional active agents selected from long acting muscarinic antagonists, long acting beta agonists, short acting beta-2 agonists, inhaled corticosteroids or a combination of two or more of them.

To assist better patient compliance, combination products are still needed. It would be highly desirable, however, to provide a combination therapy suitable to reduce bronchial inflammation, bronchial constriction and bronchial secretions in a single product or dosage form. It would also be desirable to provide such a combination product or composition in a form whereby the correct dosage of the various components is easily and safely administered.

Therefore, in a preferred embodiment of the invention, the pharmaceutical compositions comprise the drugs having amine and long acting muscarinic antagonists, or comprise the drugs having amine and long acting beta agonists, or comprise the drugs having amine and short acting beta-2 agonists, or comprise the drugs having amine and inhaled corticosteroids.

According to another embodiment, the pharmaceutical compositions comprise any of the following combinations which are suitable for administration separately, sequentially or together in effective amounts of aclidinium and tiotropium, aclidinium and glycopyrronium, aclidinum and ipratropium, aclidinum and oxitropium, glycopyrronium and tiotropium, glycopyrronium and ipratropium, glycopyrronium and oxitropium, darotropium and tiotropium, darotropium and glycopyrronium, darotropium and ipratropium, darotropium and aclidinium, darotropium and oxitropium, indacaterol and tiotropium, indacaterol and glycopyrronium, indacaterol and ipratropium, indacaterol and aclidinium, indacaterol and oxitropium, vilanterol and tiotropium, vilanterol and glycopyrronium, vilanterol and ipratropium, vilanterol and aclidinium, vilanterol and oxitropium, carmoterol and tiotropium, carmoterol and glycopyrronium, carmoterol and ipratropium, carmoterol and aclidinium, carmoterol and oxitropium, olodaterol and tiotropium, olodaterol and ipratropium, olodaterol and glycopyrronium, olodaterol and aclidinium, olodaterol and oxitropium wherein the above therapeutic agents can be present as a pharmaceutically acceptable salt or ester thereof, or in enantiomerically pure form or as a racemic mixture. According to another embodiment, the pharmaceutical compositions comprise any of the following combinations which are suitable for administration separately, sequentially or together in effective amounts of aclidinium and salmeterol, aclidinum and formoterol, aclidinium and arformoterol, aclidinium and indacaterol, aclidinium and olodaterol, aclidinium and vilanterol, aclidinium and carmoterol, aclidinium and bambuterol, glycopyrronium and salmeterol, glycopyrronium and formoterol, glycopyrronium and arformoterol, glycopyrronium and indacaterol, glycopyrronium and olodaterol, glycopyrronium and vilanterol, glycopyrronium and carmoterol, glycopyrronium and bambuterol, darotropium and salmeterol, darotropium and formoterol, darotropium and arformoterol, darotropium and indacaterol, darotropium and olodaterol, darotropium and vilanterol, darotropium and carmoterol, darotropium and bambuterol, indacaterol and salmeterol, indacaterol and formoterol, indacaterol and arformoterol, indacaterol and olodaterol, indacaterol and vilanterol, indacaterol and carmoterol, indacaterol and bambuterol, vilanterol and salmeterol, vilanterol and formoterol, vilanterol and arformoterol, vilanterol and olodaterol, vilanterol and carmoterol, vilanterol and bambuterol, carmoterol and salmeterol, carmoterol and formoterol, carmoterol and arformoterol, carmoterol and olodaterol, carmoterol and bambuterol, olodaterol and salmeterol, olodaterol and formoterol, olodaterol and arformoterol, olodaterol and bambuterol wherein the above therapeutic agents can be present as a pharmaceutically acceptable salt or ester thereof, or in enantiomerically pure form or as a racemic mixture.

According to another embodiment, the pharmaceutical compositions comprise any of the following combinations which are suitable for administration separately, sequentially or together in effective amounts of aclidinium and salbutamol, aclidinium and levosalbutamol, aclidinium and terbutaline, aclidinium and pirbuterol, aclidinium and procaterol, aclidinium and fenoterol, aclidinium and bitolterol, aclidinium and ritodrine, aclidinium and metaproterenol, glycopyrronium and salbutamol, glycopyrronium and levosalbutamol, glycopyrronium and terbutaline, glycopyrronium and pirbuterol, glycopyrronium and procaterol, glycopyrronium and fenoterol, glycopyrronium and bitolterol, glycopyrronium and ritodrine, glycopyrronium and metaproterenol, darotropium and salbutamol, darotropium and levosalbutamol, darotropium and terbutaline, darotropium and pirbuterol, darotropium and procaterol, darotropium and fenoterol, darotropium and bitolterol, darotropium and ritodrine, darotropium and metaproterenol, indacaterol and salbutamol, indacaterol and levosalbutamol, indacaterol and terbutaline, indacaterol and pirbuterol, indacaterol and procaterol, indacaterol and fenoterol, indacaterol and bitolterol, indacaterol and ritodrine, indacaterol and metaproterenol, vilanterol and salbutamol, vilanterol and levosalbutamol, vilanterol and terbutaline, vilanterol and pirbuterol, vilanterol and procaterol, vilanterol and fenoterol, vilanterol and bitolterol, vilanterol and ritodrine, vilanterol and metaproterenol, carmoterol and salbutamol, carmoterol and levosalbutamol, carmoterol and terbutaline, carmoterol and pirbuterol, carmoterol and procaterol, carmoterol and fenoterol, carmoterol and bitolterol, carmoterol and ritodrine, carmoterol and metaproterenol, olodaterol and salbutamol, olodaterol and levosalbutamol, olodaterol and terbutaline, olodaterol and pirbuterol, olodaterol and procaterol, olodaterol and fenoterol, olodaterol and bitolterol, olodaterol and ritodrine, olodaterol and metaproterenol wherein the above therapeutic agents can be present as a pharmaceutically acceptable salt or ester thereof, or in enantiomerically pure form or as a racemic mixture.

According to another embodiment, the pharmaceutical compositions comprise any of the following combinations which are suitable for administration separately, sequentially or together in effective amounts of aclidinium and fluticasone, aclidinium and ciclesonide, aclidinium and budesonide, aclidinium and mometasone, aclidinium and beclomethasone, aclidinium and triamcinolone, aclidinium and flunisolide, aclidinium and dexamethasone, glycopyrronium and fluticasone, glycopyrronium and ciclesonide, glycopyrronium and budesonide, glycopyrronium and mometasone, glycopyrronium and beclomethasone, glycopyrronium and triamcinolone, glycopyrronium and flunisolide, glycopyrronium and dexamethasone, darotropium and fluticasone, darotropium and ciclesinide, darotropium and budesonide, darotropium and mometasone, darotropium and beclomethasone, darotropium and triamcinolone, darotropium and flunisolide, darotropium and dexamethasone, indacaterol and fluticasone, indacaterol and ciclesonide, indacaterol and budesonide, indacaterol and mometasone, indacaterol and beclomethasone, indacaterol and triamcinolone, indacaterol and flunisolide, indacaterol and dexamethasone, vilanterol and fluticasone, vilanterol and ciclesonide, vilanterol and budesonide, vilanterol and mometasone, vilanterol and beclomethasone, vilanterol and triamcinolone, vilanterol and flunisolide, vilanterol and dexamethasone, carmoterol and fluticasone, carmoterol and ciclesonide, carmoterol and budesonide, carmoterol and mometasone, carmoterol and beclomethasone, carmoterol and triamcinolone, carmoterol and flunisolide, carmoterol and dexamethasone, olodaterol and fluticasone, olodaterol and ciclesonide, olodaterol and budesonide, olodaterol and mometasone, olodaterol and beclamethasone, olodaterol and triamcinolone, olodaterol and flunisolide, olodaterol and dexamethasone wherein the above therapeutic agents can be present as a pharmaceutically acceptable salt or ester thereof, or in enantiomerically pure form or as a racemic mixture.

We have also found that certain therapeutic three-in-one combinations further comprising specific, LABAs, SABAs, LAMAs and/or inhaled corticosteroids surprisingly provide an enhanced, synergistic, effect in terms of treatment of bronchoconstriction, inflammation and mucous secretions of airways. Also the three-in-one combination therapy as provided by the present invention is an extremely patient-friendly combination, which results in maximum patient compliance and better control of asthma and chronic obstructive pulmonary disease than the known combinations or single therapies.

It will also be appreciated from the above that the respective therapeutic agents of the combined preparations can be administered simultaneously, either in the same or different pharmaceutical formulations, or separately or sequentially. If there is separate or sequential administration, it will also be appreciated that the subsequently administered therapeutic agents should be administered to a patient within a time scale so as to achieve, or more particularly optimise, the above referred to advantageous synergistic therapeutic effect of a combined preparation as present in a pharmaceutical product according to the present invention.

Therefore, in a further embodiment, the pharmaceutical compositions of the invention comprise the drugs having amine, long acting muscarinic antagonists and long acting beta agonists, or comprise the drugs having amine, long acting muscarinic antagonists and short acting beta-2 agonists, or comprise the drugs having amine, long acting muscarinic antagonists and inhaled corticosteroids, or comprise the drugs having amine, long acting beta angonists and short acting beta-2 agonists, or comprise the drugs having amine, long acting beta angonists and inhaled corticosteroids, or comprise the drugs having amine, short acting beta-2 agonists and inhaled corticosteroids.

According to another embodiment, the pharmaceutical compositions comprise any of the following combinations which are suitable for administration separately, sequentially or together in effective amounts of;
i. Aclidinum, tiotropium and salmeterol
ii. Aclidinum, tiotropium and formoterol
iii. Aclidinum, tiotropium and arformoterol
iv. Aclidinum, tiotropium and indacaterol
v. Aclidinum, tiotropium and olodaterol
vi. Aclidinum, tiotropium and vilanterol
vii. Aclidinum, tiotropium and carmoterol
viii. Aclidinum, tiotropium and bambuterol
ix. Aclidinum, glycopyrronium and salmeterol
x. Aclidinum, glycopyrronium and formoterol
xi. Aclidinum, glycopyrronium and arformoterol
xii. Aclidinum, glycopyrronium and indacaterol
xiii. Aclidinum, glycopyrronium and olodaterol
xiv. Aclidinum, glycopyrronium and vilanterol
xv. Aclidinum, glycopyrronium and carmoterol
xvi. Aclidinum, glycopyrronium and bambuterol
xvii. Aclidinum, oxitropium and salmeterol
xviii. Aclidinum, oxitropium and formoterol
xix. Aclidinum, oxitropium and arformoterol
xx. Aclidinum, oxitropium and indacaterol
xxi. Aclidinum, oxitropium and olodaterol
xxii. Aclidinum, oxitropium and vilanterol
xxiii. Aclidinum, oxitropium and carmoterol
xxiv. Aclidinum, oxitropium and bambuterol
xxv. Glycopyrronium, tiotropium and salmeterol
xxvi. Glycopyrronium, tiotropium and formoterol
xxvii. Glycopyrronium, tiotropium and arformoterol
xxviii. Glycopyrronium, tiotropium and indacaterol
xxix. Glycopyrronium, tiotropium and olodaterol
xxx. Glycopyrronium, tiotropium and vilanterol
xxxi. Glycopyrronium, tiotropium and carmoterol
xxxii. Glycopyrronium, tiotropium and bambuterol
xxxiii. Glycopyrronium, oxitropium and salmeterol
xxxiv. Glycopyrronium, oxitropium and formoterol
xxxv. Glycopyrronium, oxitropium and arformoterol
xxxvi. Glycopyrronium, oxitropium and indacaterol
xxxvii. Glycopyrronium, oxitropium and olodaterol
xxxviii. Glycopyrronium, oxitropium and vilanterol
xxxix. Glycopyrronium, oxitropium and carmoterol
xl. Glycopyrronium, oxitropium and bambuterol
xli. Daratropium, tiotropium and salmeterol
xlii. Daratropium, tiotropium and formoterol
xliii. Daratropium, tiotropium and arformoterol
xliv. Daratropium, tiotropium and indacaterol
xlv. Daratropium, tiotropium and olodaterol
xlvi. Daratropium, tiotropium and vilanterol
xlvii. Daratropium, tiotropium and carmoterol
xlviii. Daratropium, tiotropium and bambuterol
xlix. Daratropium, gycopyrronium and salmeterol
l. Daratropium, gycopyrronium and formoterol
li. Daratropium, gycopyrronium and arformoterol
lii. Daratropium, gycopyrronium and indacaterol
liii. Daratropium, gycopyrronium and olodaterol
liv. Daratropium, gycopyrronium and vilanterol
lv. Daratropium, gycopyrronium and carmoterol
lvi. Daratropium, gycopyrronium and bambuterol
lvii. Daratropium, aclidinium and salmeterol
lviii. Daratropium, aclidinium and formoterol
lix. Daratropium, aclidinium and arformoterol
lx. Daratropium, aclidinium and indacaterol
lxi. Daratropium, aclidinium and olodaterol
lxii. Daratropium, aclidinium and vilanterol
lxiii. Daratropium, aclidinium and carmoterol
lxiv. Daratropium, aclidinium and bambuterol
lxv. Daratropium, oxitropium and salmeterol
lxvi. Daratropium, oxitropium and formoterol
lxvii. Daratropium, oxitropium and arformoterol
lxviii. Daratropium, oxitropium and indacaterol
lxix. Daratropium, oxitropium and olodaterol
lxx. Daratropium, oxitropium and vilanterol
lxxi. Daratropium, oxitropium and carmoterol
lxxii. Daratropium, oxitropium and bambuterol
lxxiii. Indacaterol, tirotropium and salmeterol
lxxiv. Indacaterol, tirotropium and formoterol
lxxv. Indacaterol, tirotropium and arformoterol
lxxvi. Indacaterol, tirotropium and olodaterol
lxxvii. Indacaterol, tirotropium and vilanterol
lxxviii. Indacaterol, tirotropium and carmoterol
lxxix. Indacaterol, tirotropium and bambuterol
lxxx. Indacaterol, glycopyrronium and salmeterol
lxxxi. Indacaterol, glycopyrronium and formoterol
lxxxii. Indacaterol, glycopyrronium and arformoterol
lxxxiii. Indacaterol, glycopyrronium and olodaterol
lxxxiv. Indacaterol, glycopyrronium and vilanterol
lxxxv. Indacaterol, glycopyrronium and carmoterol
lxxxvi. Indacaterol, glycopyrronium and bambuterol
lxxxvii. Indacaterol, aclidinium and salmeterol
lxxxviii. Indacaterol, aclidinium and formoterol
lxxxix. Indacaterol, aclidinium and arformoterol
xc. Indacaterol, aclidinium and olodaterol
xci. Indacaterol, aclidinium and vilanterol
xcii. Indacaterol, aclidinium and carmoterol
xciii. Indacaterol, aclidinium and bambuterol
xciv. Indacaterol, oxitropium and salmeterol
xcv. Indacaterol, oxitropium and formoterol
xcvi. Indacaterol, oxitropium and arformoterol
xcvii. Indacaterol, oxitropium and olodaterol
xcviii. Indacaterol, oxitropium and vilanterol
xcix. Indacaterol, oxitropium and carmoterol
c. Indacaterol, oxitropium and bambuterol
ci. Vilanterol, tiotropium and salmeterol
cii. Vilanterol, tiotropium and formoterol
ciii. Vilanterol, tiotropium and arformoterol
civ. Vilanterol, tiotropium and indacaterol
cv. Vilanterol, tiotropium and olodaterol
cvi. Vilanterol, tiotropium and carmoterol
cvii. Vilanterol, tiotropium and bambuterol
cviii. Vilanterol, glycopyrronium and salmeterol
cix. Vilanterol, glycopyrronium and formoterol
cx. Vilanterol, glycopyrronium and arformoterol
cxi. Vilanterol, glycopyrronium and indacaterol
cxii. Vilanterol, glycopyrronium and olodaterol
cxiii. Vilanterol, glycopyrronium and carmoterol
cxiv. Vilanterol, glycopyrronium and bambuterol
cxv. Vilanterol, aclidinium and salmeterol
cxvi. Vilanterol, aclidinium and formoterol
cxvii. Vilanterol, aclidinium and arformoterol
cxviii. Vilanterol, aclidinium and indacaterol
cxix. Vilanterol, aclidinium and olodaterol
cxx. Vilanterol, aclidinium and carmoterol
cxxi. Vilanterol, aclidinium and bambuterol
cxxii. Vilanterol, oxitropium and salmeterol
cxxiii. Vilanterol, oxitropium and formoterol
cxxiv. Vilanterol, oxitropium and arformoterol
cxxv. Vilanterol, oxitropium and indacaterol
cxxvi. Vilanterol, oxitropium and olodaterol
cxxvii. Vilanterol, oxitropium and carmoterol
cxxviii. Vilanterol, oxitropium and bambuterol
cxxix. Carmoterol, tiotropium and salmeterol
cxxx. Carmoterol, tiotropium and formoterol
cxxxi. Carmoterol, tiotropium and arformoterol
cxxxii. Carmoterol, tiotropium and indacaterol
cxxxiii. Carmoterol, tiotropium and olodaterol
cxxxiv. Carmoterol, tiotropium and vilanterol
cxxxv. Carmoterol, tiotropium and bambuterol
cxxxvi. Carmoterol, glycopyrronium and salmeterol
cxxxvii. Carmoterol, glycopyrronium and formoterol
cxxxviii. Carmoterol, glycopyrronium and arformoterol
cxxxix. Carmoterol, glycopyrronium and indacaterol
cxl. Carmoterol, glycopyrronium and olodaterol
cxli. Carmoterol, glycopyrronium and vilanterol
cxlii. Carmoterol, glycopyrronium and bambuterol
cxliii. Carmoterol, aclidinium and salmeterol
cxliv. Carmoterol, aclidinium and formoterol
cxlv. Carmoterol, aclidinium and arformoterol
cxlvi. Carmoterol, aclidinium and indacaterol
cxlvii. Carmoterol, aclidinium and olodaterol
cxlviii. Carmoterol, aclidinium and vilanterol
cxlix. Carmoterol, aclidinium and bambuterol
cl. Carmoterol, oxitropium and salmeterol
cli. Carmoterol, oxitropium and formoterol
clii. Carmoterol, oxitropium and arformoterol
cliii. Carmoterol, oxitropium and indacaterol
cliv. Carmoterol, oxitropium and olodaterol
clv. Carmoterol, oxitropium and vilanterol
clvi. Carmoterol, oxitropium and bambuterol
clvii. Olodaterol, tiotropium and salmeterol
clviii. Olodaterol, tiotropium and formoterol
clix. Olodaterol, tiotropium and arformoterol
clx. Olodaterol, tiotropium and indacaterol
clxi. Olodaterol, tiotropium and vilanterol
clxii. Olodaterol, tiotropium and bambuterol
clxiii. Olodaterol, glycopyrronium and salmeterol
clxiv. Olodaterol, glycopyrronium and formoterol
clxv. Olodaterol, glycopyrronium and arformoterol
clxvi. Olodaterol, glycopyrronium and indacaterol
clxvii. Olodaterol, glycopyrronium and vilanterol
clxviii. Olodaterol, glycopyrronium and bambuterol
clxix. Olodaterol, aclidinium and salmeterol
clxx. Olodaterol, aclidinium and formoterol
clxxi. Olodaterol, aclidinium and arformoterol
clxxii. Olodaterol, aclidinium and indacaterol
clxxiii. Olodaterol, aclidinium and vilanterol
clxxiv. Olodaterol, aclidinium and bambuterol
clxxv. Olodaterol, oxitropium and salmeterol
clxxvi. Olodaterol, oxitropium and formoterol
clxxvii. Olodaterol, oxitropium and arformoterol
clxxviii. Olodaterol, oxitropium and indacaterol
clxxix. Olodaterol, oxitropium and vilanterol
clxxx. Olodaterol, oxitropium and bambuterol
   wherein the above therapeutic agents can be present as a pharmaceutically acceptable salt or ester thereof, or in enantiomerically pure form or as a racemic mixture.

According to another embodiment, the pharmaceutical compositions comprise any of the following combinations which are suitable for administration separately, sequentially or together in effective amounts of;
i. Aclidinum, tiotropium and salbutamol
ii. Aclidinum, tiotropium and levosalbutamol
iii. Aclidinum, tiotropium and terbutaline
iv. Aclidinum, tiotropium and pirbutarol
v. Aclidinum, tiotropium and procaterol
vi. Aclidinum, tiotropium and fenoterol
vii. Aclidinum, tiotropium and ritodrine
viii. Aclidinum, tiotropium and bitolterol
ix. Aclidinum, tiotropium and metaproterenol
x. Aclidinum, glycopyrronium and salbutamol
xi. Aclidinum, glycopyrronium and levosalbutamol
xii. Aclidinum, glycopyrronium and terbutaline
xiii. Aclidinum, glycopyrronium and pirbuterol
xiv. Aclidinum, glycopyrronium and procaterol
xv. Aclidinum, glycopyrronium and fenoterol
xvi. Aclidinum, glycopyrronium and bitolterol
xvii. Aclidinum, glycopyrronium and ritodrine
xviii. Aclidinum, glycopyrronium and metaproterenol
xix. Aclidinum, ipratropium and salbutamol
xx. Aclidinum, ipratropium and levosalbutamol
xxi. Aclidinum, ipratropium and terbutaline
xxii. Aclidinum, ipratropium and pirbuterol
xxiii. Aclidinum, ipratropium and procaterol
xxiv. Aclidinum, ipratropium and fenoterol
xxv. Aclidinum, ipratropium and bitolterol
xxvi. Aclidinum, ipratropium and ritodrine
xxvii. Aclidinum, ipratropium and metaproterenol
xxviii. Aclidinum, oxitropium and salbutamol
xxix. Aclidinum, oxitropium and levosalbutamol
xxx. Aclidinum, oxitropium and terbutaline
xxxi. Aclidinum, oxitropium and pirbuterol
xxxii. Aclidinum, oxitropium and procaterol
xxxiii. Aclidinum, oxitropium and fenoterol
xxxiv. Aclidinum, oxitropium and bitolterol
xxxv. Aclidinum, oxitropium and ritodrine
xxxvi. Aclidinum, oxitropium and metaproterenol
xxxvii. Glycopyrronium, tiotropium and salbutamol
xxxviii. Glycopyrronium, tiotropium and levosalbutamol
xxxix. Glycopyrronium, tiotropium and terbutaline
xl. Glycopyrronium, tiotropium and pirbuterol
xli. Glycopyrronium, tiotropium and procaterol
xlii. Glycopyrronium, tiotropium and fenoterol
xliii. Glycopyrronium, tiotropium and bitolterol
xliv. Glycopyrronium, tiotropium and ritodrine
xlv. Glycopyrronium, tiotropium and metaproterenol
xlvi. Glycopyrronium, ipratropium and salbutamol
xlvii. Glycopyrronium, ipratropium and levosalbutamol
xlviii. Glycopyrronium, ipratropium and terbutaline
xlix. Glycopyrronium, ipratropium and pirbuterol
l. Glycopyrronium, ipratropium and procaterol
li. Glycopyrronium, ipratropium and fenoterol
lii. Glycopyrronium, ipratropium and bitolterol
liii. Glycopyrronium, ipratropium and ritodrine
liv. Glycopyrronium, ipratropium and metaproterenol
lv. Glycopyrronium, oxitropium and salbutamol
lvi. Glycopyrronium, oxitropium and levosalbutamol
lvii. Glycopyrronium, oxitropium and terbutaline
lviii. Glycopyrronium, oxitropium and pirbuterol
lix. Glycopyrronium, oxitropium and procaterol
lx. Glycopyrronium, oxitropium and fenoterol
lxi. Glycopyrronium, oxitropium and bitolterol
lxii. Glycopyrronium, oxitropium and ritodrine
lxiii. Glycopyrronium, oxitropium and metaproterenol
lxiv. Daratropium, tiotropium and salbutamol
lxv. Daratropium, tiotropium and levosalbutamol
lxvi. Daratropium, tiotropium and terbutaline
lxvii. Daratropium, tiotropium and pirbuterol
lxviii. Daratropium, tiotropium and procaterol
lxix. Daratropium, tiotropium and fenoterol
lxx. Daratropium, tiotropium and bitolterol
lxxi. Daratropium, tiotropium and ritodrine
lxxii. Daratropium, tiotropium and metaproterenol
lxxiii. Daratropium, aclidinium and salbutamol
lxxiv. Daratropium, aclidinium and levosalbutamol
lxxv. Daratropium, aclidinium and terbutaline
lxxvi. Daratropium, aclidinium and pirbuterol
lxxvii. Daratropium, aclidinium and procaterol
lxxviii. Daratropium, aclidinium and fenoterol
lxxix. Daratropium, aclidinium and bitolterol
lxxx. Daratropium, aclidinium and ritodrine
lxxxi. Daratropium, aclidinium and metaproterenol
lxxxii. Daratropium, glycopyrronium and salbutamol
lxxxiii. Daratropium, glycopyrronium and levosalbutamol
lxxxiv. Daratropium, glycopyrronium and terbutaline
lxxxv. Daratropium, glycopyrronium and pirbuterol
lxxxvi. Daratropium, glycopyrronium and procaterol
lxxxvii. Daratropium, glycopyrronium and fenoterol
lxxxviii. Daratropium, glycopyrronium and bitolterol
lxxxix. Daratropium, glycopyrronium and ritodrine
xc. Daratropium, glycopyrronium and metaproterenol
xci. Daratropium, ipratropium and salbutamol
xcii. Daratropium, ipratropium and levosalbutamol
xciii. Daratropium, ipratropium and terbutaline
xciv. Daratropium, ipratropium and pirbuterol
xcv. Daratropium, ipratropium and procaterol
xcvi. Daratropium, ipratropium and fenoterol
xcvii. Daratropium, ipratropium and bitolterol
xcviii. Daratropium, ipratropium and ritodrine
xcix. Daratropium, ipratropium and metaproterenol
c. Daratropium, oxitropium and salbutamol
ci. Daratropium, oxitropium and levosalbutamol
cii. Daratropium, oxitropium and terbutaline
ciii. Daratropium, oxitropium and pirbuterol
civ. Daratropium, oxitropium and procaterol
cv. Daratropium, oxitropium and fenoterol
cvi. Daratropium, oxitropium and bitolterol
cvii. Daratropium, oxitropium and ritodrine
cviii. Daratropium, oxitropium and metaproterenol
cix. Indacaterol, tirotropium and salbutamol
cx. Indacaterol, tirotropium and levosalbutamol
cxi. Indacaterol, tirotropium and terbutaline
cxii. Indacaterol, tirotropium and pirbuterol
cxiii. Indacaterol, tirotropium and procaterol
cxiv. Indacaterol, tirotropium and fenoterol
cxv. Indacaterol, tirotropium and bitolterol
cxvi. Indacaterol, tirotropium and ritodrine
cxvii. Indacaterol, tirotropium and metaproterenol
cxviii. Indacaterol, glycopyrronium and salbutamol
cxix. Indacaterol, glycopyrronium and levosalbutamol
cxx. Indacaterol, glycopyrronium and terbutaline
cxxi. Indacaterol, glycopyrronium and pirbuterol
cxxii. Indacaterol, glycopyrronium and procaterol
cxxiii. Indacaterol, glycopyrronium and fenoterol
cxxiv. Indacaterol, glycopyrronium and bitolterol
cxxv. Indacaterol, glycopyrronium and ritodrine
cxxvi. Indacaterol, glycopyrronium and metaproterenol
cxxvii. Indacaterol, aclidinium and salbutamol
cxxviii. Indacaterol, aclidinium and levosalbutamol
cxxix. Indacaterol, aclidinium and terbutaline
cxxx. Indacaterol, aclidinium and pirbuterol
cxxxi. Indacaterol, aclidinium and procaterol
cxxxii. Indacaterol, aclidinium and fenoterol
cxxxiii. Indacaterol, aclidinium and bitolterol
cxxxiv. Indacaterol, aclidinium and ritodrine
cxxxv. Indacaterol, aclidinium and metaproterenol
cxxxvi. Indacaterol, ipratropium and salbutamol
cxxxvii. Indacaterol, ipratropium and levosalbutamol
cxxxviii. Indacaterol, ipratropium and terbutaline
cxxxix. Indacaterol, ipratropium and pirbuterol
cxl. Indacaterol, ipratropium and procaterol
cxli. Indacaterol, ipratropium and fenoterol
cxlii. Indacaterol, ipratropium and bitolterol
cxliii. Indacaterol, ipratropium and ritodrine
cxliv. Indacaterol, ipratropium and metaproterenol
cxlv. Indacaterol, oxitropium and salbutamol
cxlvi. Indacaterol, oxitropium and levosalbutamol
cxlvii. Indacaterol, oxitropium and terbutaline
cxlviii. Indacaterol, oxitropium and pirbuterol
cxlix. Indacaterol, oxitropium and procaterol
cl. Indacaterol, oxitropium and fenoterol
cli. Indacaterol, oxitropium and bitolterol
clii. Indacaterol, oxitropium and ritodrine
cliii. Indacaterol, oxitropium and metaproterenol
cliv. Vilanterol, tiotropium and salbutamol
clv. Vilanterol, tiotropium and levosalbutamol
clvi. Vilanterol, tiotropium and terbutaline
clvii. Vilanterol, tiotropium and pirbuterol
clviii. Vilanterol, tiotropium and procaterol
clix. Vilanterol, tiotropium and fenoterol
clx. Vilanterol, tiotropium and bitolterol
clxi. Vilanterol, tiotropium and ritodrine
clxii. Vilanterol, tiotropium and metaproterenol
clxiii. Vilanterol, glycopyrronium and salbutamol
clxiv. Vilanterol, glycopyrronium and levosalbutamol
clxv. Vilanterol, glycopyrronium and terbutaline
clxvi. Vilanterol, glycopyrronium and pirbuterol
clxvii. Vilanterol, glycopyrronium and procaterol
clxviii. Vilanterol, glycopyrronium and fenoterol
clxix. Vilanterol, glycopyrronium and bitolterol
clxx. Vilanterol, glycopyrronium and ritodrine
clxxi. Vilanterol, glycopyrronium and metaproterenol
clxxii. Vilanterol, ipratropium and salbutamol
clxxiii. Vilanterol, ipratropium and levosalbutamol
clxxiv. Vilanterol, ipratropium and terbutaline
clxxv. Vilanterol, ipratropium and pirbuterol
clxxvi. Vilanterol, ipratropium and procaterol
clxxvii. Vilanterol, ipratropium and fenoterol
clxxviii. Vilanterol, ipratropium and bitolterol
clxxix. Vilanterol, ipratropium and ritodrine
clxxx. Vilanterol, ipratropium and metaproterenol
clxxxi. Vilanterol, aclidinium and salbutamol
clxxxii. Vilanterol, aclidinium and levosalbutamol
clxxxiii. Vilanterol, aclidinium and terbutaline
clxxxiv. Vilanterol, aclidinium and pirbuterol
clxxxv. Vilanterol, aclidinium and procaterol
clxxxvi. Vilanterol, aclidinium and fenoterol
clxxxvii. Vilanterol, aclidinium and bitolterol
clxxxviii. Vilanterol, aclidinium and ritodrine
clxxxix. Vilanterol, aclidinium and metaproterenol
cxc. Vilanterol, oxitropium and salbutamol
cxci. Vilanterol, oxitropium and levosalbutamol
cxcii. Vilanterol, oxitropium and terbutaline
cxciii. Vilanterol, oxitropium and pirbuterol
cxciv. Vilanterol, oxitropium and procaterol
cxcv. Vilanterol, oxitropium and fenoterol
cxcvi. Vilanterol, oxitropium and bitolterol
cxcvii. Vilanterol, oxitropium and ritodrine
cxcviii. Vilanterol, oxitropium and metaproterenol
cxcix. Carmoterol, tiotropium and salbutamol
cc. Carmoterol, tiotropium and levosalbutamol
cci. Carmoterol, tiotropium and terbutaline
ccii. Carmoterol, tiotropium and pirbuterol
cciii. Carmoterol, tiotropium and procaterol
cciv. Carmoterol, tiotropium and fenoterol
ccv. Carmoterol, tiotropium and bitolterol
ccvi. Carmoterol, tiotropium and ritodrine
ccvii. Carmoterol, tiotropium and metaproterenol
ccviii. Carmoterol, ipratropium and levosalbutamol
ccix. Carmoterol, ipratropium and salbutamol
ccx. Carmoterol, ipratropium and terbutaline
ccxi. Carmoterol, ipratropium and pirbuterol
ccxii. Carmoterol, ipratropium and procaterol
ccxiii. Carmoterol, ipratropium and fenoterol
ccxiv. Carmoterol, ipratropium and bitolterol
ccxv. Carmoterol, ipratropium and ritodrine
ccxvi. Carmoterol, ipratropium and metaproterenol
ccxvii. Carmoterol, aclidinum and levosalbutamol
ccxviii. Carmoterol, aclidinum and salbutamol
ccxix. Carmoterol, aclidinum and terbutaline
ccxx. Carmoterol, aclidinum and pirbuterol
ccxxi. Carmoterol, aclidinum and procaterol
ccxxii. Carmoterol, aclidinum and fenoterol
ccxxiii. Carmoterol, aclidinum and bitolterol
ccxxiv. Carmoterol, aclidinum and ritodrine
ccxxv. Carmoterol, aclidinum and metaproterenol
ccxxvi. Carmoterol, oxitropium and salbutamol
ccxxvii. Carmoterol, oxitropium and levosalbutamol
ccxxviii. Carmoterol, oxitropium and terbutaline
ccxxix. Carmoterol, oxitropium and pirbuterol
ccxxx. Carmoterol, oxitropium and procaterol
ccxxxi. Carmoterol, oxitropium and fenoterol
ccxxxii. Carmoterol, oxitropium and bitolterol
ccxxxiii. Carmoterol, oxitropium and ritodrine
ccxxxiv. Carmoterol, oxitropium and metaproterenol
ccxxxv. Olodaterol, tiotropium and salbutamol
ccxxxvi. Olodaterol, tiotropium and levosalbutamol
ccxxxvii. Olodaterol, tiotropium and terbutaline
ccxxxviii. Olodaterol, tiotropium and pirbuterol
ccxxxix. Olodaterol, tiotropium and procaterol
ccxl. Olodaterol, tiotropium and fenoterol
ccxli. Olodaterol, tiotropium and bitolterol
ccxlii. Olodaterol, tiotropium and ritodrine
ccxliii. Olodaterol, tiotropium and metaproterenol
ccxliv. Olodaterol, ipratropium and salbutamol
ccxlv. Olodaterol, ipratropium and levosalbutamol
ccxlvi. Olodaterol, ipratropium and terbutaline
ccxlvii. Olodaterol, ipratropium and pirbuterol
ccxlviii. Olodaterol, ipratropium and procaterol
ccxlix. Olodaterol, ipratropium and fenoterol
ccl. Olodaterol, ipratropium and bitolterol
ccli. Olodaterol, ipratropium and ritodrine
cclii. Olodaterol, ipratropium and metaproterenol
ccliii. Olodaterol, aclidinum and salbutamol
ccliv. Olodaterol, aclidinum and levosalbutamol
cclv. Olodaterol, aclidinum and terbutaline
cclvi. Olodaterol, aclidinum and pirbuterol
cclvii. Olodaterol, aclidinum and procaterol
cclviii. Olodaterol, aclidinum and fenoterol
cclix. Olodaterol, aclidinum and bitolterol
cclx. Olodaterol, aclidinum and ritodrine
cclxi. Olodaterol, aclidinum and metaproterenol
cclxii. Olodaterol, glycopyrronium and salbutamol
cclxiii. Olodaterol, glycopyrronium and levosalbutamol
cclxiv. Olodaterol, glycopyrronium and terbutaline
cclxv. Olodaterol, glycopyrronium and pirbuterol
cclxvi. Olodaterol, glycopyrronium and procaterol
cclxvii. Olodaterol, glycopyrronium and fenoterol
cclxviii. Olodaterol, glycopyrronium and bitolterol
cclxix. Olodaterol, glycopyrronium and ritodrine
cclxx. Olodaterol, glycopyrronium and metaproterenol
cclxxi. Olodaterol, oxitropium and salbutamol
cclxxii. Olodaterol, oxitropium and levosalbutamol
cclxxiii. Olodaterol, oxitropium and terbutaline
cclxxiv. Olodaterol, oxitropium and pirbuterol
cclxxv. Olodaterol, oxitropium and procaterol
cclxxvi. Olodaterol, oxitropium and fenoterol
cclxxvii. Olodaterol, oxitropium and bitolterol
cclxxviii. Olodaterol, oxitropium and ritodrine
cclxxix. Olodaterol, oxitropium and metaproterenol
wherein the above therapeutic agents can be present as a pharmaceutically acceptable salt or ester thereof, or in enantiomerically pure form or as a racemic mixture.

According to another embodiment, the pharmaceutical compositions comprise any of the following combinations which are suitable for administration separately, sequentially or together in effective amounts of;
i. Aclidinum, tiotropium and fluticasone
ii. Aclidinum, tiotropium and ciclesonide
iii. Aclidinum, tiotropium and budesonide
iv. Aclidinum, tiotropium and mometasone
v. Aclidinum, tiotropium and beclomethasone
vi. Aclidinum, tiotropium and triamcinolone
vii. Aclidinum, tiotropium and flunisolide
viii. Aclidinum, tiotropium and dexomethasone
ix. Daratropium, tiotropium and fluticasone
x. Daratropium, tiotropium and ciclesonide
xi. Daratropium, tiotropium and budesonide
xii. Daratropium, tiotropium and mometasone
xiii. Daratropium, tiotropium and beclamethasone
xiv. Daratropium, tiotropium and triamcinolone
xv. Daratropium, tiotropium and flunisolide
xvi. Daratropium, tiotropium and dexomethasone
xvii. Indacaterol, tiotropium and fluticasone
xviii. Indacaterol, tiotropium and budesonide
xix. Indacaterol, tiotropium and ciclesonide
xx. Indacaterol, tiotropium and mometasone
xxi. Indacaterol, tiotropium and beclamethasone
xxii. Indacaterol, tiotropium and triamcinolone
xxiii. Indacaterol, tiotropium and flunisolide
xxiv. Indacaterol, tiotropium and dexomethasone
xxv. Vilanterol, tiotropium and ciclesonide
xxvi. vilanterol, tiotropium and fluticasone
xxvii. vilanterol, tiotropium and budesonide
xxviii. vilanterol, tiotropium and mometasone
xxix. vilanterol, tiotropium and beclamethasone
xxx. vilanterol, tiotropium and triamcinolone
xxxi. vilanterol, tiotropium and flunisolide
xxxii. vilanterol, tiotropium and dexomethasone
xxxiii. carmoterol, tiotropium and budesonide
xxxiv. carmoterol, tiotropium and ciclesonide
xxxv. carmoterol, tiotropium and fluticasone
xxxvi. carmoterol, tiotropium and mometasone
xxxvii. carmoterol, tiotropium and beclamethasone
xxxviii. carmoterol, tiotropium and triamcinolone
xxxix. carmoterol, tiotropium and flunisolide
xl. carmoterol, tiotropium and dexomethasone
xli. Olodaterol, tiotropium and ciclesonide
xlii. Olodaterol, tiotropium and fluticasone
xliii. Olodaterol, tiotropium and budesonide
xliv. Olodaterol, tiotropium and mometasone
xlv. Olodaterol, tiotropium and beclamethasone
xlvi. Olodaterol, tiotropium and triamcinolone
xlvii. Olodaterol, tiotropium and flunisolide
xlviii. Olodaterol, tiotropium and dexomethasone
   wherein the above therapeutic agents can be present as a pharmaceutically acceptable salt or ester thereof, or in enantiomerically pure form or as a racemic mixture.

According to another embodiment, the pharmaceutical compositions comprise any of the following combinations which are suitable for administration separately, sequentially or together in effective amounts of;
i. Aclidinium, salmeterol and salbutamol
ii. Aclidinium, salmeterol and levosalbutamol
iii. Aclidinium, formoterol and salbutamol
iv. Aclidinium, formoterol and levosalbutamol
v. Aclidinium, arformoterol and salbutamol
vi. Aclidinium, arformoterol and levosalbutamol
vii. Aclidinium, indacaterol and salbutamol
viii. Aclidinium, indacaterol and levosalbutamol
ix. Aclidinium, olodaterol and salbutamol
x. Aclidinium, olodaterol and levosalbutamol
xi. Aclidinium, vilanterol and salbutamol
xii. Aclidinium, vilanterol and levosalbutamol
xiii. Aclidinium, carmoterol and salbutamol
xiv. Aclidinium, carmoterol and levosalbutamol
xv. Aclidinium, bambuterol and salbutamol
xvi. Aclidinium, bambuterol and levosalbutamol
xvii. Glycopyrronium, indacaterol and salbutamol
xviii. Glycopyrronium, indacaterol and levosalbutamol
xix. Glycopyrronium, salmeterol and salbutamol
xx. Glycopyrronium, salmeterol and levosalbutamol
xxi. Glycopyrronium, formoterol and salbutamol
xxii. Glycopyrronium, formoterol and levosalbutamol
xxiii. Glycopyrronium, arformoterol and salbutamol
xxiv. Glycopyrronium, arformoterol and levosalbutamol
xxv. Glycopyrronium, carmoterol and salbutamol
xxvi. Glycopyrronium, carmoterol and levosalbutamol
xxvii. Glycopyrronium, olodaterol and salbutamol
xxviii. Glycopyrronium, olodaterol and levosalbutamol
xxix. Glycopyrronium, vilanterol and salbutamol
xxx. Glycopyrronium, vilanterol and levosalbutamol
xxxi. Glycopyrronium, bambuterol and salbutamol
xxxii. Glycopyrronium, bambuterol and levosalbutamol
xxxiii. Daratropium, indacaterol and salbutamol
xxxiv. Daratropium, indacaterol and levosalbutamol
xxxv. Daratropium, salmeterol and salbutamol
xxxvi. Daratropium, salmeterol and levosalbutamol
xxxvii. Daratropium, formoterol and salbutamol
xxxviii. Daratropium, formoterol and levosalbutamol
xxxix. Daratropium, carmoterol and salbutamol
xl. Daratropium, carmoterol and levosalbutamol
xli. Daratropium, olodaterol and salbutamol
xlii. Daratropium, olodaterol and levosalbutamol
xliii. Daratropium, vilanterol and salbutamol
xliv. Daratropium, vilanterol and levosalbutamol
xlv. Daratropium, bambuterol and salbutamol
xlvi. Daratropium, bambuterol and levosalbutamol
xlvii. Daratropium, arformoterol and salbutamol
xlviii. Daratropium, arformoterol and levosalbutamol
   wherein the above therapeutic agents can be present as a pharmaceutically acceptable salt or ester thereof, or in enantiomerically pure form or as a racemic mixture.

According to another embodiment, the pharmaceutical compositions comprise any of the following combinations which are suitable for administration separately, sequentially or together in effective amounts of;
i. Aclidinium, salmeterol and mometasone
ii. Aclidinium, salmeterol and fluticasone
iii. Aclidinium, salmeterol and budesonide
iv. Aclidinium, formoterol and mometasone
v. Aclidinium, formoterol and fluticasone
vi. Aclidinium, formoterol and budesonide
vii. Aclidinium, arformoterol and mometasone
viii. Aclidinium, arformoterol and fluticasone
ix. Aclidinium, arformoterol and budesonide
x. Aclidinium, indacaterol and mometasone
xi. Aclidinium, indacaterol and fluticasone
xii. Aclidinium, indacaterol and budesonide
xiii. Aclidinium, olodaterol and mometasone
xiv. Aclidinium, olodaterol and fluticasone
xv. Aclidinium, olodaterol and budesonide
xvi. Aclidinium, vilanterol and mometasone
xvii. Aclidinium, vilanterol and fluticasone
xviii. Aclidinium, vilanterol and budesonide
xix. Aclidinium, carmoterol and mometasone
xx. Aclidinium, carmoterol and fluticasone
xxi. Aclidinium, carmoterol and budesonide
xxii. Aclidinium, bambuterol and mometasone
xxiii. Aclidinium, bambuterol and fluticasone
xxiv. Aclidinium, bambuterol and budesonide
xxv. Glycopyrronium, indacaterol and mometasone
xxvi. Glycopyrronium, indacaterol and fluticasone
xxvii. Glycopyrronium, indacaterol and budesonide
xxviii. Glycopyrronium, salmeterol and mometasone
xxix. Glycopyrronium, salmeterol and fluticasone
xxx. Glycopyrronium, salmeterol and budesonide
xxxi. Glycopyrronium, formoterol and mometasone
xxxii. Glycopyrronium, formoterol and fluticasone
xxxiii. Glycopyrronium, formoterol and budesonide
xxxiv. Glycopyrronium, arformoterol and mometasone
xxxv. Glycopyrronium, arformoterol and fluticasone
xxxvi. Glycopyrronium, arformoterol and budesonide
xxxvii. Glycopyrronium, carmoterol and mometasone
xxxviii. Glycopyrronium, carmoterol and fluticasone
xxxix. Glycopyrronium, carmoterol and budesonide
xl. Glycopyrronium, olodaterol and mometasone
xli. Glycopyrronium, olodaterol and fluticasone
xlii. Glycopyrronium, olodaterol and budesonide
xliii. Glycopyrronium, vilanterol and mometasone
xliv. Glycopyrronium, vilanterol and fluticasone
xlv. Glycopyrronium, vilanterol and budesonide
xlvi. Glycopyrronium, bambuterol and mometasone
xlvii. Glycopyrronium, bambuterol and fluticasone
xlviii. Glycopyrronium, bambuterol and budesonide
xlix. Daratropium, indacaterol and mometasone
l. Daratropium, indacaterol and fluticasone
li. Daratropium, indacaterol and budesonide
lii. Daratropium, salmeterol and mometasone
liii. Daratropium, salmeterol and fluticasone
liv. Daratropium, salmeterol and budesonide
lv. Daratropium, formoterol and mometasone
lvi. Daratropium, formoterol and fluticasone
lvii. Daratropium, formoterol and budesonide
lviii. Daratropium, carmoterol and mometasone
lix. Daratropium, carmoterol and fluticasone
lx. Daratropium, carmoterol and budesonide
lxi. Daratropium, olodaterol and mometasone
lxii. Daratropium, olodaterol and fluticasone
lxiii. Daratropium, olodaterol and budesonide
lxiv. Daratropium, vilanterol and mometasone
lxv. Daratropium, vilanterol and fluticasone
lxvi. Daratropium, vilanterol and budesonide
lxvii. Daratropium, bambuterol and mometasone
lxviii. Daratropium, bambuterol and fluticasone
lxix. Daratropium, bambuterol and budesonide
lxx. Daratropium, arformoterol and mometasone
lxxi. Daratropium, arformoterol and fluticasone
lxxii. Daratropium, arformoterol and budesonide
lxxiii. Indacaterol, salmeterol and mometasone
lxxiv. Indacaterol, salmeterol and fluticasone
lxxv. Indacaterol, salmeterol and budesonide
lxxvi. Indacaterol, formoterol and mometasone
lxxvii. Indacaterol, formoterol and fluticasone
lxxviii. Indacaterol, formoterol and budesonide
lxxix. Indacaterol, arformoterol and mometasone
lxxx. Indacaterol, arformoterol and fluticasone
lxxxi. Indacaterol, arformoterol and budesonide
lxxxii. Indacaterol, olodaterol and mometasone
lxxxiii. Indacaterol, olodaterol and fluticasone
lxxxiv. Indacaterol, olodaterol and budesonide
lxxxv. Indacaterol, vilanterol and mometasone
lxxxvi. Indacaterol, vilanterol and fluticasone
lxxxvii. Indacaterol, vilanterol and budesonide
lxxxviii. Indacaterol, carmeterol and mometasone
lxxxix. Indacaterol, carmeterol and fluticasone
xc. Indacaterol, carmeterol and budesonide
xci. Indacaterol, bambuterol and mometasone
xcii. Indacaterol, bambuterol and fluticasone
xciii. Indacaterol, bambuterol and budesonide
xciv. Vilanterol, salmeterol and mometasone
xcv. Vilanterol, salmeterol and fluticasone
xcvi. Vilanterol, salmeterol and budesonide
xcvii. Vilanterol, formoterol and mometasone
xcviii. Vilanterol, formoterol and fluticasone
xcix. Vilanterol, formoterol and budesonide
c. Vilanterol, arformoterol and mometasone
ci. Vilanterol, arformoterol and fluticasone
cii. Vilanterol, arformoterol and budesonide
ciii. Vilanterol, olodaterol and mometasone
civ. Vilanterol, olodaterol and fluticasone
cv. Vilanterol, olodaterol and budesonide
cvi. Vilanterol, carmeterol and mometasone
cvii. Vilanterol, carmeterol and fluticasone
cviii. Vilanterol, carmeterol and budesonide
cix. Vilanterol, bambuterol and mometasone
cx. Vilanterol, bambuterol and fluticasone
cxi. Vilanterol, bambuterol and budesonide
cxii. Vilanterol, indacaterol and mometasone
cxiii. Vilanterol, indacaterol and fluticasone
cxiv. Vilanterol, indacaterol and budesonide
cxv. Carmeterol, salmeterol and mometasone
cxvi. Carmeterol, salmeterol and fluticasone
cxvii. Carmeterol, salmeterol and budesonide
cxviii. Carmeterol, formoterol and mometasone
cxix. Carmeterol, formoterol and fluticasone
cxx. Carmeterol, formoterol and budesonide
cxxi. Carmeterol, arformoterol and mometasone
cxxii. Carmeterol, arformoterol and fluticasone
cxxiii. Carmeterol, arformoterol and budesonide
cxxiv. Carmeterol, indaceterol and mometasone
cxxv. Carmeterol, indaceterol and fluticasone
cxxvi. Carmeterol, indaceterol and budesonide
cxxvii. Carmeterol, olodaterol and mometasone
cxxviii. Carmeterol, olodaterol and fluticasone
cxxix. Carmeterol, olodaterol and budesonide
cxxx. Carmeterol, vilanterol and mometasone
cxxxi. Carmeterol, vilanterol and fluticasone
cxxxii. Carmeterol, vilanterol and budesonide
cxxxiii. Carmeterol, bambuterol and mometasone
cxxxiv. Carmeterol, bambuterol and fluticasone
cxxxv. Carmeterol, bambuterol and budesonide
cxxxvi. Olodaterol, salmeterol and mometasone
cxxxvii. Olodaterol, salmeterol and fluticasone
cxxxviii. Olodaterol, salmeterol and budesonide
cxxxix. Olodaterol, formoterol and mometasone
cxl. Olodaterol, formoterol and fluticasone
cxli. Olodaterol, formoterol and budesonide
cxlii. Olodaterol, arformoterol and mometasone
cxliii. Olodaterol, arformoterol and fluticasone
cxliv. Olodaterol, arformoterol and budesonide
cxlv. Olodaterol, indaceterol and mometasone
cxlvi. Olodaterol, indaceterol and fluticasone
cxlvii. Olodaterol, indaceterol and budesonide
cxlviii. Olodaterol, vilanterol and mometasone
cxlix. Olodaterol, vilanterol and fluticasone
cl. Olodaterol, vilanterol and budesonide
cli. Olodaterol, carmeterol and mometasone
clii. Olodaterol, carmeterol and fluticasone
cliii. Olodaterol, carmeterol and budesonide
cliv. Olodaterol, bambuterol and mometasone
clv. Olodaterol, bambuterol and fluticasone
clvi. Olodaterol, bambuterol and budesonide
   wherein the above therapeutic agents can be present as a pharmaceutically acceptable salt or ester thereof, or in enantiomerically pure form or as a racemic mixture.

According to another embodiment, the pharmaceutical compositions comprise any of the following combinations which are suitable for administration separately, sequentially or together in effective amounts of;
i. Aclidinium, salbutamol and fluticasone
ii. Aclidinium, levosalbutamol and fluticasone
iii. Aclidinium, salbutamol and ciclesonide
iv. Aclidinium, levosalbutamol and ciclesonide
v. Aclidinium, salbutamol and budesonide
vi. Aclidinium, levosalbutamol and budesonide
vii. Aclidinium, salbutamol and mometasone
viii. Aclidinium, levosalbutamol and mometasone
ix. Aclidinium, salbutamol and beclometahsone
x. Aclidinium, levosalbutamol and beclometahsone
xi. Aclidinium, salbutamol and triamcinolone
xii. Aclidinium, levosalbutamol and triamcinolone
xiii. Aclidinium, salbutamol and flunisolide
xiv. Aclidinium, levosalbutamol and flunisolide
xv. Aclidinium, salbutamol and dexamethasone
xvi. Aclidinium, levosalbutamol and dexamethasone
xvii. Glycopyrronium, salbutamol and fluticasone
xviii. Glycopyrronium, levosalbutamol and fluticasone
xix. Glycopyrronium, salbutamol and ciclesonide
xx. Glycopyrronium, levosalbutamol and ciclesonide
xxi. Glycopyrronium, salbutamol and budesonide
xxii. Glycopyrronium, levosalbutamol and budesonide
xxiii. Glycopyrronium, salbutamol and mometasone
xxiv. Glycopyrronium, levosalbutamol and mometasone
xxv. Glycopyrronium, salbutamol and beclometahsone
xxvi. Glycopyrronium, levosalbutamol and beclometahsone
xxvii. Glycopyrronium, salbutamol and triamcinolone
xxviii. Glycopyrronium, levosalbutamol and triamcinolone
xxix. Glycopyrronium, salbutamol and flunisolide
xxx. Glycopyrronium, levosalbutamol and flunisolide
xxxi. Glycopyrronium, salbutamol and dexamethasone
xxxii. Glycopyrronium, levosalbutamol and dexamethasone
xxxiii. Daratropium, salbutamol and fluticasone
xxxiv. Daratropium, levosalbutamol and fluticasone
xxxv. Daratropium, salbutamol and ciclesonide
xxxvi. Daratropium, levosalbutamol and ciclesonide
xxxvii. Daratropium, salbutamol and budesonide
xxxviii. Daratropium, levosalbutamol and budesonide
xxxix. Daratropium, salbutamol and mometasone
xl. Daratropium, levosalbutamol and mometasone
xli. Daratropium, salbutamol and beclometahsone
xlii. Daratropium, levosalbutamol and beclometahsone
xliii. Daratropium, salbutamol and triamcinolone
xliv. Daratropium, levosalbutamol and triamcinolone
xlv. Daratropium, salbutamol and flunisolide
xlvi. Daratropium, levosalbutamol and flunisolide
xlvii. Daratropium, salbutamol and dexamethasone
xlviii. Daratropium, levosalbutamol and dexamethasone
xlix. Indacaterol, salbutamol and fluticasone
l. Indacaterol, levosalbutamol and fluticasone
li. Indacaterol, salbutamol and ciclesonide
lii. Indacaterol, levosalbutamol and ciclesonide
liii. Indacaterol, salbutamol and budesonide
liv. Indacaterol, levosalbutamol and budesonide
lv. Indacaterol, salbutamol and mometasone
lvi. Indacaterol, levosalbutamol and mometasone
lvii. Indacaterol, salbutamol and beclometahsone
lviii. Indacaterol, levosalbutamol and beclometahsone
lix. Indacaterol, salbutamol and triamcinolone
lx. Indacaterol, levosalbutamol and triamcinolone
lxi. Indacaterol, salbutamol and flunisolide
lxii. Indacaterol, levosalbutamol and flunisolide
lxiii. Indacaterol, salbutamol and dexamethasone
lxiv. Indacaterol, levosalbutamol and dexamethasone
lxv. Vilanterol, salbutamol and fluticasone
lxvi. Vilanterol, levosalbutamol and fluticasone
lxvii. Vilanterol, salbutamol and budesonide
lxviii. Vilanterol, levosalbutamol and budesonide
lxix. Vilanterol, salbutamol and ciclesonide
lxx. Vilanterol, levosalbutamol and ciclesonide
lxxi. Vilanterol, salbutamol and mometasone
lxxii. Vilanterol, levosalbutamol and mometasone
lxxiii. Vilanterol, salbutamol and beclomethasone
lxxiv. Vilanterol, levosalbutamol and beclomethasone
lxxv. Vilanterol, salbutamol and triamcinolone
lxxvi. Vilanterol, levosalbutamol and triamcinolone
lxxvii. Vilanterol, salbutamol and flunisolide
lxxviii. Vilanterol, levosalbutamol and flunisolide
lxxix. Vilanterol, salbutamol and dexamethasone
lxxx. Vilanterol, levosalbutamol and dexamethasone
lxxxi. Carmeterol, salbutamol and fluticasone
lxxxii. Carmeterol, levosalbutamol and fluticasone
lxxxiii. Carmeterol, salbutamol and ciclesonide
lxxxiv. Carmeterol, levosalbutamol and ciclesonide
lxxxv. Carmeterol, salbutamol and budesonide
lxxxvi. Carmeterol, levosalbutamol and budesonide
lxxxvii. Carmeterol, salbutamol and mometasone
lxxxviii. Carmeterol, levosalbutamol and mometasone
lxxxix. Carmeterol, salbutamol and beclomethasone
xc. Carmeterol, levosalbutamol and beclomethasone
xci. Carmeterol, salbutamol and triamcinolone
xcii. Carmeterol, levosalbutamol and triamcinolone
xciii. Carmeterol, salbutamol and flunisolide
xciv. Carmeterol, levosalbutamol and flunisolide
xcv. Carmeterol, salbutamol and dexamethasone
xcvi. Carmeterol, levosalbutamol and dexamethasone
xcvii. Olodaterol, salbutamol and fluticasone
xcviii. Olodaterol, levosalbutamol and fluticasone
xcix. Olodaterol, salbutamol and ciclesonide
c. Olodaterol, levosalbutamol and ciclesonide
ci. Olodaterol, salbutamol and budesonide
cii. Olodaterol, levosalbutamol and budesonide
ciii. Olodaterol, salbutamol and mometasone
civ. Olodaterol, levosalbutamol and mometasone
cv. Olodaterol, salbutamol and beclomethasone
cvi. Olodaterol, levosalbutamol and beclomethasone
cvii. Olodaterol, salbutamol and triamcinolone
cviii. Olodaterol, levosalbutamol and triamcinolone
cix. Olodaterol, salbutamol and flunisolide
cx. Olodaterol, levosalbutamol and flunisolide
cxi. Olodaterol, salbutamol and dexamethasone
cxii. Olodaterol, levosalbutamol and dexamethasone
   wherein the above therapeutic agents can be present as a pharmaceutically acceptable salt or ester thereof, or in enantiomerically pure form or as a racemic mixture.

According to another embodiment, the pharmaceutical compositions comprise any of the following combinations which are suitable for administration separately, sequentially or together in effective amounts of;
i. Formoterol, budesonide and tiotropium
ii. Salmeterol, fluticasone and tiotropium
iii. Carmoterol, tiotropium and fluticasone
iv. Salbutamol, formoterol and budesonide
v. Salbutamol, salmeterol and fluticasone
vi. Salbutamol, arformoterol and fluticasone
   wherein the above therapeutic agents can be present as a pharmaceutically acceptable salt or ester thereof, or in enantiomerically pure form or as a racemic mixture.

According to a preferred embodiment of the invention, the therapeutically effective amount of said pharmaceutical compositions are administered once a day and/or administered twice a day.

According to a preferred embodiment, the pharmaceutical compositions are used for in the treatment of respiratory conditions selected from asthma and chronic obstructive pulmonary disease and other obstructive airways diseases. In particular, the combinations of compounds of the present invention are useful in the treatment of respiratory diseases and conditions comprising, asthma, acute respiratory distress syndrome, chronic pulmonary inflammatory disease, bronchitis, chronic bronchitis, chronic obstructive pulmonary (airway) disease, and silicosis; or immune diseases and conditions comprising: allergic rhinitis and chronic sinusitis.

According to a further embodiment, the pharmaceutical compositions are suitable for administration separately, sequentially or together in effective amounts, together with a moisture tight and high barrier sealed blister or together with a capsule

In particular, the blister comprises aluminium to prevent ingress of moisture whereby the fine particle fraction (FPF) of the pharmaceutical composition dose is preserved. Furthermore, the blister is a high barrier sealed against moisture. Thus, the blister does not release any water to the dose and ingress of moisture from the exterior into the container is thereby prevented.

In a further preferred embodiment of the invention, the dry powder is in a capsule, which can be a pharmaceutically acceptable natural or synthetic polymer such as gelatin or hydroxypropyl methylcellulose.

In a preferred embodiment, the pharmaceutical compositions are suitable for administration separately, sequentially or together in effective amounts, together with an inhalation device. The device is preferably dry powder inhaler including the blister or the capsule described above.

In a further embodiment, the device in which the pharmaceutical composition is within the blister comprise at least one lock mechanism, enabling the device to remain locked in both positions in which the device is ready for inhalation and the lid is in the closed position, and further enabling the device to setup again automatically, when the lid is closed.

In a further embodiment, the invention relates to a pharmaceutical kit comprising the drugs having amine and one or more additional active agents, in separate unit dosage forms, said forms being suitable for administration separately, sequentially or together in effective amounts, together with one or more inhalation devices for administration of drugs having amine and one or more additional active agents which are LAMAs, LABAs, SABAs and/or inhaled corticosteroids as described in detail above.

In a further embodiment, the process for making the pharmaceutical compositions for inhalation of the present invention comprises the following steps;

To obtain a homogenous mixture first half of the coarse mannitol particles are added to a glass container later on fine mannitol particles are added and active ingredients are added to this mixture and blended in a turbula shaker. Then this mixture is elected. This election is not a milling, the aim of this election is to obtain a homogenous mixture. Then magnesium stearate and the rest of the coarse mannitol particles are added to this elected mixture during blending. Final powder mixture is furthermore blended and then filled into blisters or capsules.

This invention is further defined by reference to the following examples. In the following examples the drugs having amine has the ratio between the median particle size (d₅₀) and d₉₀ is about 0.50. Although these examples are not intended to limit the scope of the present invention, it should be considered in the light of the description detailed above.

### EXAMPLE-1

TABLE 1

| Ingredients | Amount % (w/w) |
|---|---|
| drugs having amine | 0.01-10.0 |
| fine mannitol | 2.0-20.0 |
| coarse mannitol | 20.0-98.0 |
| Magnesium stearate | 0.05-1.0 |

Particle size of the drugs having amine (μm):

| | | |
|---|---|---|
| d₁₀: 0.10-1.0 | d₅₀: 1.0-2.5 | d₉₀: 2.5-5.0 |

Particle size of the fine mannitol (μm):

| | | |
|---|---|---|
| d₁₀: 1.0-4.0 | d₅₀: 4.0-7.0 | d₉₀: 7.0-15.0 |

Particle size of the coarse mannitol (μm):

| | | |
|---|---|---|
| d₁₀: 10-50 | d₅₀: 50-75 | d₉₀: 75-250 |

Particle size of the magnesium stearate (μm):

| | | |
|---|---|---|
| d₁₀: 0.25-2.5 | d₅₀: 3.0-7.0 | d₉₀: 7.0-20.0 |

### EXAMPLE-2

TABLE 2

| Ingredients | Amount % (w/w) |
|---|---|
| drugs having amine | 0.01-10.0 |
| LABAs | 0.01-10.0 |
| fine mannitol | 2.0-20.0 |
| coarse mannitol | 20.0-98.0 |
| Magnesium stearate | 0.05-1.0 |

### Particle Size of;

| | | | |
|---|---|---|---|
| LABAs (μm): | d₁₀: 0.10-1.0 | d₅₀: 1.0-2.5 | d₉₀: 2.5-5.0 |
| the drugs having amine (μm): | d₁₀: 0.10-1.0 | d₅₀: 1.0-2.5 | d₉₀: 2.5-5.0 |
| fine mannitol (μm): | d₁₀: 1.0-4.0 | d₅₀: 4.0-7.0 | d₉₀: 7.0-15.0 |
| coarse mannitol (μm): | d₁₀: 10-50 | d₅₀: 50-75 | d₉₀: 75-250 |
| magnesium stearate (μm): | d₁₀: 0.25-2.5 | d₅₀: 3.0-7.0 | d₉₀: 7.0-20.0 |

### EXAMPLES-3

TABLE 3

| Ingredients | Amount % (w/w) |
|---|---|
| drugs having amine | 0.01-10.0 |
| LAMAs | 0.01-10.0 |
| fine mannitol | 2.0-20.0 |
| coarse mannitol | 20.0-98.0 |
| magnesium stearate | 0.05-1.0 |

### Particle Size of;

| | | | |
|---|---|---|---|
| the drugs having amine (μm): | d₁₀: 0.10-1.0 | d₅₀: 1.0-2.5 | d₉₀: 2.5-5.0 |
| LAMAs(μm): | d₁₀: 0.10-1.0 | d₅₀: 1.0-2.5 | d₉₀: 2.5-5.0 |
| fine mannitol (μm): | d₁₀: 1.0-4.0 | d₅₀: 4.0-7.0 | d₉₀: 7.0-15.0 |
| coarse mannitol (μm): | d₁₀: 10-50 | d₅₀: 50-75 | d₉₀: 75-250 |
| magnesium stearate (μm): | d₁₀: 0.25-2.5 | d₅₀: 3.0-7.0 | d₉₀: 7.0-20.0 |

### EXAMPLES-4

TABLE 4

| Ingredients | Amount % (w/w) |
|---|---|
| drugs having amine | 0.01-10.0 |
| SABAs | 0.01-10.0 |
| fine mannitol | 2.0-20.0 |
| coarse mannitol | 20.0-98.0 |
| magnesium stearate | 0.05-1.0 |

### Particle Size of;

| | | | |
|---|---|---|---|
| SABAs (μm): | d₁₀: 0.10-1.0 | d₅₀: 1.0-2.5 | d₉₀: 2.5-5.0 |
| the drugs having amine (μm): | d₁₀: 0.10-1.0 | d₅₀: 1.0-2.5 | d₉₀: 2.5-5.0 |
| fine mannitol (μm): | d₁₀: 1.0-4.0 | d₅₀: 4.0-7.0 | d₉₀: 7.0-15.0 |
| coarse mannitol (μm): | d₁₀: 10-50 | d₅₀: 50-75 | d₉₀: 75-250 |
| magnesium stearate (μm): | d₁₀: 0.25-2.5 | d₅₀: 3.0-7.0 | d₉₀: 7.0-20.0 |

### EXAMPLES-5

TABLE 5

| Ingredients | Amount % (w/w) |
|---|---|
| drugs having amine | 0.01-10.0 |
| Corticosteroids | 0.01-10.0 |
| fine mannitol | 2.0-20.0 |
| coarse mannitol | 20.0-98.0 |
| magnesium stearate | 0.05-1.0 |

### Particle Size of;

| | | | |
|---|---|---|---|
| Corticosteroids(μm): | d₁₀: 0.1-1.0 | d₅₀: 1.0-2.5 | d₉₀: 2.5-5.0 |
| the drugs having amine (μm): | d₁₀: 0.10-1.0 | d₅₀: 1.0-2.5 | d₉₀: 2.5-5.0 |
| fine mannitol (μm): | d₁₀: 1.0-4.0 | d₅₀: 4.0-7.0 | d₉₀: 7.0-15.0 |
| coarse mannitol (μm): | d₁₀: 10-50 | d₅₀: 50-75 | d₉₀: 75-250 |
| magnesium stearate (μm): | d₁₀: 0.25-2.5 | d₅₀: 3.0-7.0 | d₉₀: 7.0-20.0 |

### EXAMPLES-6

Particle size of each actives, mannitol and magnesium stearate are the same as above examples 1 to 5.

TABLE 6.1

| Ingredients | Amount % (w/w) |
|---|---|
| drugs having amine | 0.01-10.0 |
| LAMAs | 0.01-10.0 |
| LABAs | 0.01-10.0 |
| fine mannitol | 2.0-20.0 |
| coarse mannitol | 20.0-98.0 |
| magnesium stearate | 0.05-1.0 |

TABLE 6.2

| Ingredients | Amount % (w/w) |
|---|---|
| drugs having amine | 0.01-10.0 |
| LAMAs | 0.01-10.0 |
| SABAs | 0.01-10.0 |
| fine mannitol | 2.0-20.0 |
| coarse mannitol | 20.0-98.0 |
| magnesium stearate | 0.05-1.0 |

TABLE 6.3

| Ingredients | Amount % (w/w) |
|---|---|
| drugs having amine | 0.01-10.0 |
| LAMAs | 0.01-10.0 |
| corticosteroids | 0.01-10.0 |
| fine mannitol | 2.0-20.0 |
| coarse mannitol | 20.0-98.0 |
| magnesium stearate | 0.05-1.0 |

TABLE 6.4

| Ingredients | Amount % (w/w) |
|---|---|
| drugs having amine | 0.01-10.0 |
| LABAs | 0.01-10.0 |
| SABAs | 0.01-10.0 |
| fine mannitol | 2.0-20.0 |
| coarse mannitol | 20.0-98.0 |
| magnesium stearate | 0.05-1.0 |

TABLE 6.5

| Ingredients | Amount % (w/w) |
|---|---|
| drugs having amine | 0.01-10.0 |
| LABAs | 0.01-10.0 |
| corticosteroids | 0.01-10.0 |
| fine mannitol | 2.0-20.0 |
| coarse mannitol | 20.0-98.0 |
| magnesium stearate | 0.05-1.0 |

TABLE 6.6

| Ingredients | Amount % (w/w) |
|---|---|
| drugs having amine | 0.01-10.0 |
| SABAs | 0.01-10.0 |
| corticosteroids | 0.01-10.0 |
| fine mannitol | 2.0-20.0 |
| coarse mannitol | 20.0-98.0 |
| magnesium stearate | 0.05-1.0 |

1. A pharmaceutical composition for inhalation comprising separately, sequentially or together, drugs having amine in the form of a dry powder in admixture with a pharmaceutically acceptable carrier, other than lactose and at least one ternary component.

2. The pharmaceutical composition according to claim 1, wherein the ternary component is selected from a group comprising magnesium stearate, stearic acid, sodium lauryl sulphate, sodium stearyl fumarate, stearyl alcohol and sodium benzoate or mixtures thereof.

3. The pharmaceutical composition according to claim 2, wherein the ternary component is magnesium stearate.

4. The pharmaceutical composition according to claim 3, wherein magnesium stearate is in an amount of 0.05 to 2.0% by weight, preferably in an amount of 0.1 to 1.0% by weight, more preferably in an amount of 0.15 to 0.5% by weight.

5. The pharmaceutical composition according to claim 3, wherein the particles of the magnesium stearate have a particle diameter of d₁₀ between 0.25-2.5 μm, d₅₀ between 3.0-7.0 μm and d₉₀ between 7.0-20.0 μm.

6. The pharmaceutical composition according to claim 1, wherein the pharmaceutically acceptable carrier, other than lactose, comprise fine and coarse particles and the ratio between the fine particles to the coarse particles is between 0.01-0.25 by weight.

7. The pharmaceutical composition according to claim 1, wherein the fine particles of the said pharmaceutically acceptable carrier have a particle diameter of d₁₀ between 1.0-4.0 μm, d₅₀ between 4.0-7.0 μm and d₉₀ between 7.0-15.0 μm.

8. The pharmaceutical composition according to claim 1, wherein the coarse particles of the said pharmaceutically acceptable carrier have a particle diameter of d₁₀ between 10-50 μm, d₅₀ between 50-75 μm and d₉₀ between 75-250 μm.

9. The pharmaceutical composition according to claim 1, wherein the pharmaceutically acceptable carrier is selected from the group comprising mannitol, glucose, trehalose, cellobiose, sorbitol, maltitol or a combination of two or more of them.

10. The pharmaceutical composition according to claim 9, wherein the pharmaceutically acceptable carrier is preferably mannitol.

11. The pharmaceutical composition according to claim 10, wherein the pharmaceutically acceptable carrier is optionally spray dried mannitol.

12. The pharmaceutical composition according to claim 1, wherein the average particle diameter (d₅₀) of the drugs having amine is between 1.5-2.5 μm.

13. The pharmaceutical composition according to claim 1, wherein the amine is primary amine and/or secondary amine.

14. The pharmaceutical composition according to claim 1, wherein the drug is aclidinium or a pharmaceutically acceptable salt or ester thereof, or in enantiomerically pure form or as a racemic mixture.

15. The pharmaceutical composition according to claim 1, wherein the drug is glycopyrronium or a pharmaceutically acceptable salt or ester thereof, or in enantiomerically pure form or as a racemic mixture.

16. The pharmaceutical composition according to claim 13, wherein the drug is darotropium or a pharmaceutically acceptable salt or ester thereof, or in enantiomerically pure form or as a racemic mixture.

17. The pharmaceutical composition according to claim 13, wherein the drug is indacaterol or a pharmaceutically acceptable salt or ester thereof, or in enantiomerically pure form or as a racemic mixture.

18. The pharmaceutical composition according to claim 13, wherein the drug is vilanterol or a pharmaceutically acceptable salt or ester thereof, or in enantiomerically pure form or as a racemic mixture.

19. The pharmaceutical composition according to claim 13, wherein the drug is carmoterol or a pharmaceutically acceptable salt or ester thereof, or in enantiomerically pure form or as a racemic mixture.

20. The pharmaceutical composition according to claim 13, wherein the drug is olodaterol or a pharmaceutically acceptable salt or ester thereof, or in enantiomerically pure form or as a racemic mixture.

21. The pharmaceutical composition according to claim 1, further comprising one or more additional active agents selected from long acting muscarinic antagonists, long acting beta agonists, short acting beta-2 agonists, corticosteroids or a combination of two or more of them.

22. The pharmaceutical composition according to claim 21, comprising the drugs having amine and long acting muscarinic antagonists.

23. The pharmaceutical composition according to claim 21, comprising the drugs having amine and long acting beta agonists.

24. The pharmaceutical composition according to claim 21, comprising the drugs having amine and short acting beta-2 agonists.

25. The pharmaceutical composition according to claim 21, comprising the drugs having amine and corticosteroids.

26. The pharmaceutical composition according to claim 21, comprising the drugs having amine, long acting muscarinic antagonists and long acting beta agonists.

27. The pharmaceutical composition according to claim 21, comprising the drugs having amine, long acting muscarinic antagonists and short acting beta-2 agonists.

28. The pharmaceutical composition according to claim 21, comprising the drugs having amine, long acting muscarinic antagonists and corticosteroids.

29. The pharmaceutical composition according to claim 21, comprising the drugs having amine, long acting beta angonists and short acting beta-2 agonists.

30. The pharmaceutical composition according to claim 21, comprising the drugs having amine, long acting beta angonists and corticosteroids.

31. The pharmaceutical composition according to claim 21, comprising the drugs having amine, short acting beta-2 agonists and corticosteroids.

32. The pharmaceutical composition according to claim 21, wherein the long acting muscarinic antagonists are selected from the group comprising tiotropium, glycopyrronium, ipratropium, aclidinium, oxitropium or a pharmaceutically acceptable salt or ester thereof, or in enantiomerically pure form or as a racemic mixture or a combination of two or more of them.

33. The pharmaceutical composition according to claim 21, wherein the long acting beta agonists are selected from the group comprising salmeterol, formoterol, arformoterol, indacaterol, olodaterol, vilanterol, carmoterol, bambuterol or a pharmaceutically acceptable salt or ester thereof, or in enantiomerically pure form or as a racemic mixture or a combination of two or more of them.

34. The pharmaceutical composition according to claim 21, wherein the short acting beta-2 agonists are selected from the group comprising salbutamol, levosalbutamol, terbutaline, pirbuterol, procaterol, fenoterol, bitolterol, ritodrine, metaproterenol or a pharmaceutically acceptable salt or ester thereof, or in enantiomerically pure form or as a racemic mixture or a combination of two or more of them.

35. The pharmaceutical composition according to claim 21, wherein the corticosteroids are selected from the group comprising fluticasone, ciclesonide, budesonide, mometasone, beclomethasone, triamcinolone, flunisolide, dexamethasone or a pharmaceutically acceptable salt or ester thereof, or in enantiomerically pure form or as a racemic mixture or a combination of two or more of them.

36. The pharmaceutical composition according to claim 22, comprising any of the following combinations, said combinations being suitable for administration separately, sequentially or together in effective amounts;
i. Aclidinium and tiotropium
ii. Aclidinium and glycopyrronium
iii. Aclidinum and ipratropium
iv. Aclidinum and oxitropium
v. Glycopyrronium and tiotropium
vi. Glycopyrronium and ipratropium
vii. Glycopyrronium and oxitropium
viii. Darotropium and tiotropium
ix. Darotropium and glycopyrronium
x. Darotropium and ipratropium
xi. Darotropium and aclidinium
xii. Darotropium and oxitropium
xiii. Indacaterol and tiotropium
xiv. Indacaterol and glycopyrronium
xv. Indacaterol and ipratropium
xvi. Indacaterol and aclidinium
xvii. Indacaterol and oxitropium
xviii. Vilanterol and tiotropium
xix. Vilanterol and glycopyrronium
xx. Vilanterol and ipratropium
xxi. Vilanterol and aclidinium
xxii. Vilanterol and oxitropium
xxiii. Carmoterol and tiotropium
xxiv. Carmoterol and glycopyrronium
xxv. Carmoterol and ipratropium
xxvi. Carmoterol and aclidinium
xxvii. Carmoterol and oxitropium
xxviii. Olodaterol and tiotropium
xxix. Olodaterol and ipratropium
xxx. Olodaterol and glycopyrronium
xxxi. Olodaterol and aclidinium
xxxii. Olodaterol and oxitropium
wherein the above therapeutic agents can be present as a pharmaceutically acceptable salt or ester thereof, or in enantiomerically pure form or as a racemic mixture.

37. The pharmaceutical composition according to claim 23, comprising any of the following combinations, said combinations being suitable for administration separately, sequentially or together in effective amounts;
i. Aclidinium and salmeterol
ii. Aclidinum and formoterol
iii. Aclidinium and arformoterol
iv. Aclidinium and indacaterol
v. Aclidinium and olodaterol
vi. Aclidinium and vilanterol
vii. Aclidinium and carmoterol
viii. Aclidinium and bambuterol
ix. Glycopyrronium and salmeterol
x. Glycopyrronium and formoterol
xi. Glycopyrronium and arformoterol
xii. Glycopyrronium and indacaterol
xiii. Glycopyrronium and olodaterol
xiv. Glycopyrronium and vilanterol
xv. Glycopyrronium and carmoterol
xvi. Glycopyrronium and bambuterol
xvii. Darotropium and salmeterol
xviii. Darotropium and formoterol
xix. Darotropium and arformoterol
xx. Darotropium and indacaterol
xxi. Darotropium and olodaterol
xxii. Darotropium and vilanterol
xxiii. Darotropium and carmoterol
xxiv. Darotropium and bambuterol
xxv. Indacaterol and salmeterol
xxvi. Indacaterol and formoterol
xxvii. Indacaterol and arformoterol
xxviii. Indacaterol and olodaterol
xxix. Indacaterol and vilanterol
xxx. Indacaterol and carmoterol
xxxi. Indacaterol and bambuterol
xxxii. Vilanterol and salmeterol
xxxiii. Vilanterol and formoterol
xxxiv. Vilanterol and arformoterol
xxxv. Vilanterol and olodaterol
xxxvi. Vilanterol and carmoterol
xxxvii. Vilanterol and bambuterol
xxxviii. Carmoterol and salmeterol
xxxix. Carmoterol and formoterol
xl. Carmoterol and arformoterol
xli. Carmoterol and olodaterol
xlii. Carmoterol and bambuterol
xliii. Olodaterol and salmeterol
xliv. Olodaterol and formoterol
xlv. Olodaterol and arformoterol
xlvi. Olodaterol and bambuterol
wherein the above therapeutic agents can be present as a pharmaceutically acceptable salt or ester thereof, or in enantiomerically pure form or as a racemic mixture.

38. The pharmaceutical composition according to claim 24, comprising any of the following combinations, said combinations being suitable for administration separately, sequentially or together in effective amounts;
i. Aclidinium and salbutamol
ii. Aclidinium and levosalbutamol
iii. Aclidinium and terbutaline
iv. Aclidinium and pirbuterol
v. Aclidinium and procaterol
vi. Aclidinium and fenoterol
vii. Aclidinium and bitolterol
viii. Aclidinium and ritodrine
ix. Aclidinium and metaproterenol
x. Glycopyrronium and salbutamol
xi. Glycopyrronium and levosalbutamol
xii. Glycopyrronium and terbutaline
xiii. Glycopyrronium and pirbuterol
xiv. Glycopyrronium and procaterol
xv. Glycopyrronium and fenoterol
xvi. Glycopyrronium and bitolterol
xvii. Glycopyrronium and ritodrine
xviii. Glycopyrronium and metaproterenol
xix. Darotropium and salbutamol
xx. Darotropium and levosalbutamol
xxi. Darotropium and terbutaline
xxii. Darotropium and pirbuterol
xxiii. Darotropium and procaterol
xxiv. Darotropium and fenoterol
xxv. Darotropium and bitolterol
xxvi. Darotropium and ritodrine
xxvii. Darotropium and metaproterenol
xxviii. Indacaterol and salbutamol
xxix. Indacaterol and levosalbutamol
xxx. Indacaterol and terbutaline
xxxi. Indacaterol and pirbuterol
xxxii. Indacaterol and procaterol
xxxiii. Indacaterol and fenoterol
xxxiv. Indacaterol and bitolterol
xxxv. Indacaterol and ritodrine
xxxvi. Indacaterol and metaproterenol
xxxvii. Vilanterol and salbutamol
xxxviii. Vilanterol and levosalbutamol
xxxix. Vilanterol and terbutaline
xl. Vilanterol and pirbuterol
xli. Vilanterol and procaterol
xlii. Vilanterol and fenoterol
xliii. Vilanterol and bitolterol
xliv. Vilanterol and ritodrine
xlv. Vilanterol and metaproterenol
xlvi. Carmoterol and salbutamol
xlvii. carmoterol and levosalbutamol
xlviii. carmoterol and terbutaline
xlix. carmoterol and pirbuterol
l. carmoterol and procaterol
li. carmoterol and fenoterol
lii. carmoterol and bitolterol
liii. carmoterol and ritodrine
liv. carmoterol and metaproterenol
lv. olodaterol and salbutamol
lvi. olodaterol and levosalbutamol
lvii. olodaterol and terbutaline
lviii. olodaterol and pirbuterol
lix. olodaterol and procaterol
lx. olodaterol and fenoterol
lxi. olodaterol and bitolterol
lxii. olodaterol and ritodrine
lxiii. olodaterol and metaproterenol
wherein the above therapeutic agents can be present as a pharmaceutically acceptable salt or ester thereof, or in enantiomerically pure form or as a racemic mixture.

39. The pharmaceutical composition according to claim 25, comprising any of the following combinations, said combinations being suitable for administration separately, sequentially or together in effective amounts;
i. Aclidinium and fluticasone
ii. Aclidinium and ciclesonide
iii. Aclidinium and budesonide
iv. Aclidinium and mometasone
v. Aclidinium and beclomethasone
vi. Aclidinium and triamcinolone
vii. Aclidinium and flunisolide
viii. Aclidinium and dexamethasone
ix. Glycopyrronium and fluticasone
x. Glycopyrronium and ciclesonide
xi. Glycopyrronium and budesonide
xii. Glycopyrronium and mometasone
xiii. Glycopyrronium and beclomethasone
xiv. Glycopyrronium and triamcinolone
xv. Glycopyrronium and flunisolide
xvi. Glycopyrronium and dexamethasone
xvii. Darotropium and fluticasone
xviii. Darotropium and ciclesonide
xix. Darotropium and budesonide
xx. Darotropium and mometasone
xxi. Darotropium and beclomethasone
xxii. Darotropium and triamcinolone
xxiii. Darotropium and flunisolide
xxiv. Darotropium and dexamethasone
xxv. Indacaterol and fluticasone
xxvi. Indacaterol and ciclesonide
xxvii. Indacaterol and budesonide
xxviii. Indacaterol and mometasone
xxix. Indacaterol and beclomethasone
xxx. Indacaterol and triamcinolone
xxxi. Indacaterol and flunisolide
xxxii. Indacaterol and dexamethasone
xxxiii. Vilanterol and fluticasone
xxxiv. Vilanterol and ciclesonide
xxxv. Vilanterol and budesonide
xxxvi. Vilanterol and mometasone
xxxvii. Vilanterol and beclomethasone
xxxviii. Vilanterol and triamcinolone
xxxix. Vilanterol and flunisolide
xl. Vilanterol and dexamethasone
xli. Carmoterol and fluticasone
xlii. Carmoterol and ciclesonide
xliii. Carmoterol and budesonide
xliv. Carmoterol and mometasone
xlv. Carmoterol and beclomethasone
xlvi. Carmoterol and triamcinolone
xlvii. Carmoterol and flunisolide
xlviii. Carmoterol and dexamethasone
xlix. Olodaterol and fluticasone
l. Olodaterol and ciclesonide
li. Olodaterol and budesonide
lii. Olodaterol and mometasone
liii. Olodaterol and beclamethasone
liv. Olodaterol and triamcinolone
lv. Olodaterol and flunisolide
lvi. Olodaterol and dexamethasone
wherein the above therapeutic agents can be present as a pharmaceutically acceptable salt or ester thereof, or in enantiomerically pure form or as a racemic mixture.

40. The pharmaceutical composition according to claims 26, comprising any of the following combinations, said combinations being suitable for administration separately, sequentially or together in effective amounts;
i. Aclidinum, tiotropium and salmeterol
ii. Aclidinum, tiotropium and formoterol
iii. Aclidinum, tiotropium and arformoterol
iv. Aclidinum, tiotropium and indacaterol
v. Aclidinum, tiotropium and olodaterol
vi. Aclidinum, tiotropium and vilanterol
vii. Aclidinum, tiotropium and carmoterol
viii. Aclidinum, tiotropium and bambuterol
ix. Aclidinum, glycopyrronium and salmeterol
x. Aclidinum, glycopyrronium and formoterol
xi. Aclidinum, glycopyrronium and arformoterol
xii. Aclidinum, glycopyrronium and indacaterol
xiii. Aclidinum, glycopyrronium and olodaterol
xiv. Aclidinum, glycopyrronium and vilanterol
xv. Aclidinum, glycopyrronium and carmoterol
xvi. Aclidinum, glycopyrronium and bambuterol
xvii. Aclidinum, oxitropium and salmeterol
xviii. Aclidinum, oxitropium and formoterol
xix. Aclidinum, oxitropium and arformoterol
xx. Aclidinum, oxitropium and indacaterol
xxi. Aclidinum, oxitropium and olodaterol
xxii. Aclidinum, oxitropium and vilanterol
xxiii. Aclidinum, oxitropium and carmoterol
xxiv. Aclidinum, oxitropium and bambuterol
xxv. Glycopyrronium, tiotropium and salmeterol
xxvi. Glycopyrronium, tiotropium and formoterol
xxvii. Glycopyrronium, tiotropium and arformoterol
xxviii. Glycopyrronium, tiotropium and indacaterol
xxix. Glycopyrronium, tiotropium and olodaterol
xxx. Glycopyrronium, tiotropium and vilanterol
xxxi. Glycopyrronium, tiotropium and carmoterol
xxxii. Glycopyrronium, tiotropium and bambuterol
xxxiii. Glycopyrronium, oxitropium and salmeterol
xxxiv. Glycopyrronium, oxitropium and formoterol
xxxv. Glycopyrronium, oxitropium and arformoterol
xxxvi. Glycopyrronium, oxitropium and indacaterol
xxxvii. Glycopyrronium, oxitropium and olodaterol
xxxviii. Glycopyrronium, oxitropium and vilanterol
xxxix. Glycopyrronium, oxitropium and carmoterol
xl. Glycopyrronium, oxitropium and bambuterol
xli. Daratropium, tiotropium and salmeterol
xlii. Daratropium, tiotropium and formoterol
xliii. Daratropium, tiotropium and arformoterol
xliv. Daratropium, tiotropium and indacaterol
xlv. Daratropium, tiotropium and olodaterol
xlvi. Daratropium, tiotropium and vilanterol
xlvii. Daratropium, tiotropium and carmoterol
xlviii. Daratropium, tiotropium and bambuterol
xlix. Daratropium, glycopyrronium and salmeterol
l. Daratropium, glycopyrronium and formoterol
li. Daratropium, glycopyrronium and arformoterol
lii. Daratropium, glycopyrronium and indacaterol
liii. Daratropium, glycopyrronium and olodaterol
liv. Daratropium, glycopyrronium and vilanterol
lv. Daratropium, glycopyrronium and carmoterol
lvi. Daratropium, glycopyrronium and bambuterol
lvii. Daratropium, aclidinium and salmeterol
lviii. Daratropium, aclidinium and formoterol
lix. Daratropium, aclidinium and arformoterol
lx. Daratropium, aclidinium and indacaterol
lxi. Daratropium, aclidinium and olodaterol
lxii. Daratropium, aclidinium and vilanterol
lxiii. Daratropium, aclidinium and carmoterol
lxiv. Daratropium, aclidinium and bambuterol
lxv. Daratropium, oxitropium and salmeterol
lxvi. Daratropium, oxitropium and formoterol
lxvii. Daratropium, oxitropium and arformoterol
lxviii. Daratropium, oxitropium and indacaterol
lxix. Daratropium, oxitropium and olodaterol
lxx. Daratropium, oxitropium and vilanterol
lxxi. Daratropium, oxitropium and carmoterol
lxxii. Daratropium, oxitropium and bambuterol
lxxiii. Indacaterol, tiotropium and salmeterol
lxxiv. Indacaterol, tiotropium and formoterol
lxxv. Indacaterol, tiotropium and arformoterol
lxxvi. Indacaterol, tiotropium and olodaterol
lxxvii. Indacaterol, tiotropium and vilanterol
lxxviii. Indacaterol, tiotropium and carmoterol
lxxix. Indacaterol, tiotropium and bambuterol
lxxx. Indacaterol, glycopyrronium and salmeterol
lxxxi. Indacaterol, glycopyrronium and formoterol
lxxxii. Indacaterol, glycopyrronium and arformoterol
lxxxiii. Indacaterol, glycopyrronium and olodaterol
lxxxiv. Indacaterol, glycopyrronium and vilanterol
lxxxv. Indacaterol, glycopyrronium and carmoterol
lxxxvi. Indacaterol, glycopyrronium and bambuterol
lxxxvii. Indacaterol, aclidinium and salmeterol
lxxxviii. Indacaterol, aclidinium and formoterol
lxxxix. Indacaterol, aclidinium and arformoterol
xc. Indacaterol, aclidinium and olodaterol
xci. Indacaterol, aclidinium and vilanterol
xcii. Indacaterol, aclidinium and carmoterol
xciii. Indacaterol, aclidinium and bambuterol
xciv. Indacaterol, oxitropium and salmeterol
xcv. Indacaterol, oxitropium and formoterol
xcvi. Indacaterol, oxitropium and arformoterol
xcvii. Indacaterol, oxitropium and olodaterol
xcviii. Indacaterol, oxitropium and vilanterol
xcix. Indacaterol, oxitropium and carmoterol
c. Indacaterol, oxitropium and bambuterol
ci. Vilanterol, tiotropium and salmeterol
cii. Vilanterol, tiotropium and formoterol
ciii. Vilanterol, tiotropium and arformoterol
civ. Vilanterol, tiotropium and indacaterol
cv. Vilanterol, tiotropium and olodaterol
cvi. Vilanterol, tiotropium and carmoterol
cvii. Vilanterol, tiotropium and bambuterol
cviii. Vilanterol, glycopyrronium and salmeterol
cix. Vilanterol, glycopyrronium and formoterol
cx. Vilanterol, glycopyrronium and arformoterol
cxi. Vilanterol, glycopyrronium and indacaterol
cxii. Vilanterol, glycopyrronium and olodaterol
cxiii. Vilanterol, glycopyrronium and carmoterol
cxiv. Vilanterol, glycopyrronium and bambuterol
cxv. Vilanterol, aclidinium and salmeterol
cxvi. Vilanterol, aclidinium and formoterol
cxvii. Vilanterol, aclidinium and arformoterol
cxviii. Vilanterol, aclidinium and indacaterol
cxix. Vilanterol, aclidinium and olodaterol
cxx. Vilanterol, aclidinium and carmoterol
cxxi. Vilanterol, aclidinium and bambuterol
cxxii. Vilanterol, oxitropium and salmeterol
cxxiii. Vilanterol, oxitropium and formoterol
cxxiv. Vilanterol, oxitropium and arformoterol
cxxv. Vilanterol, oxitropium and indacaterol
cxxvi. Vilanterol, oxitropium and olodaterol
cxxvii. Vilanterol, oxitropium and carmoterol
cxxviii. Vilanterol, oxitropium and bambuterol
cxxix. Carmoterol, tiotropium and salmeterol
cxxx. Carmoterol, tiotropium and formoterol
cxxxi. Carmoterol, tiotropium and arformoterol
cxxxii. Carmoterol, tiotropium and indacaterol
cxxxiii. Carmoterol, tiotropium and olodaterol
cxxxiv. Carmoterol, tiotropium and vilanterol
cxxxv. Carmoterol, tiotropium and bambuterol
cxxxvi. Carmoterol, glycopyrronium and salmeterol
cxxxvii. Carmoterol, glycopyrronium and formoterol
cxxxviii. Carmoterol, glycopyrronium and arformoterol
cxxxix. Carmoterol, glycopyrronium and indacaterol
cxl. Carmoterol, glycopyrronium and olodaterol
cxli. Carmoterol, glycopyrronium and vilanterol
cxlii. Carmoterol, glycopyrronium and bambuterol
cxliii. Carmoterol, aclidinium and salmeterol
cxliv. Carmoterol, aclidinium and formoterol
cxlv. Carmoterol, aclidinium and arformoterol
cxlvi. Carmoterol, aclidinium and indacaterol
cxlvii. Carmoterol, aclidinium and olodaterol
cxlviii. Carmoterol, aclidinium and vilanterol
cxlix. Carmoterol, aclidinium and bambuterol
cl. Carmoterol, oxitropium and salmeterol
cli. Carmoterol, oxitropium and formoterol
clii. Carmoterol, oxitropium and arformoterol
cliii. Carmoterol, oxitropium and indacaterol
cliv. Carmoterol, oxitropium and olodaterol
clv. Carmoterol, oxitropium and vilanterol
clvi. Carmoterol, oxitropium and bambuterol
clvii. Olodaterol, tiotropium and salmeterol
clviii. Olodaterol, tiotropium and formoterol
clix. Olodaterol, tiotropium and arformoterol
clx. Olodaterol, tiotropium and indacaterol
clxi. Olodaterol, tiotropium and vilanterol
clxii. Olodaterol, tiotropium and bambuterol
clxiii. Olodaterol, glycopyrronium and salmeterol
clxiv. Olodaterol, glycopyrronium and formoterol
clxv. Olodaterol, glycopyrronium and arformoterol
clxvi. Olodaterol, glycopyrronium and indacaterol
clxvii. Olodaterol, glycopyrronium and vilanterol
clxviii. Olodaterol, glycopyrronium and bambuterol
clxix. Olodaterol, aclidinium and salmeterol
clxx. Olodaterol, aclidinium and formoterol
clxxi. Olodaterol, aclidinium and arformoterol
clxxii. Olodaterol, aclidinium and indacaterol
clxxiii. Olodaterol, aclidinium and vilanterol
clxxiv. Olodaterol, aclidinium and bambuterol
clxxv. Olodaterol, oxitropium and salmeterol
clxxvi. Olodaterol, oxitropium and formoterol
clxxvii. Olodaterol, oxitropium and arformoterol
clxxviii. Olodaterol, oxitropium and indacaterol
clxxix. Olodaterol, oxitropium and vilanterol
clxxx. Olodaterol, oxitropium and bambuterol
wherein the above therapeutic agents can be present as a pharmaceutically acceptable salt or ester thereof, or in enantiomerically pure form or as a racemic mixture.

41. The pharmaceutical composition according to claims 27, comprising any of the following combinations, said combinations being suitable for administration separately, sequentially or together in effective amounts;
i. Aclidinum, tiotropium and salbutamol
ii. Aclidinum, tiotropium and levosalbutamol
iii. Aclidinum, tiotropium and terbutaline
iv. Aclidinum, tiotropium and pirbutarol
v. Aclidinum, tiotropium and procaterol
vi. Aclidinum, tiotropium and fenoterol
vii. Aclidinum, tiotropium and ritodrine
viii. Aclidinum, tiotropium and bitolterol
ix. Aclidinum, tiotropium and metaproterenol
x. Aclidinum, glycopyrronium and salbutamol
xi. Aclidinum, glycopyrronium and levosalbutamol
xii. Aclidinum, glycopyrronium and terbutaline
xiii. Aclidinum, glycopyrronium and pirbuterol
xiv. Aclidinum, glycopyrronium and procaterol
xv. Aclidinum, glycopyrronium and fenoterol
xvi. Aclidinum, glycopyrronium and bitolterol
xvii. Aclidinum, glycopyrronium and ritodrine
xviii. Aclidinum, glycopyrronium and metaproterenol
xix. Aclidinum, ipratropium and salbutamol
xx. Aclidinum, ipratropium and levosalbutamol
xxi. Aclidinum, ipratropium and terbutaline
xxii. Aclidinum, ipratropium and pirbuterol
xxiii. Aclidinum, ipratropium and procaterol
xxiv. Aclidinum, ipratropium and fenoterol
xxv. Aclidinum, ipratropium and bitolterol
xxvi. Aclidinum, ipratropium and ritodrine
xxvii. Aclidinum, ipratropium and metaproterenol
xxviii. Aclidinum, oxitropium and salbutamol
xxix. Aclidinum, oxitropium and levosalbutamol
xxx. Aclidinum, oxitropium and terbutaline
xxxi. Aclidinum, oxitropium and pirbuterol
xxxii. Aclidinum, oxitropium and procaterol
xxxiii. Aclidinum, oxitropium and fenoterol
xxxiv. Aclidinum, oxitropium and bitolterol
xxxv. Aclidinum, oxitropium and ritodrine
xxxvi. Aclidinum, oxitropium and metaproterenol
xxxvii. Glycopyrronium, tiotropium and salbutamol
xxxviii. Glycopyrronium, tiotropium and levosalbutamol
xxxix. Glycopyrronium, tiotropium and terbutaline
xl. Glycopyrronium, tiotropium and pirbuterol
xli. Glycopyrronium, tiotropium and procaterol
xlii. Glycopyrronium, tiotropium and fenoterol
xliii. Glycopyrronium, tiotropium and bitolterol
xliv. Glycopyrronium, tiotropium and ritodrine
xlv. Glycopyrronium, tiotropium and metaproterenol
xlvi. Glycopyrronium, ipratropium and salbutamol
xlvii. Glycopyrronium, ipratropium and levosalbutamol
xlviii. Glycopyrronium, ipratropium and terbutaline
xlix. Glycopyrronium, ipratropium and pirbuterol
l. Glycopyrronium, ipratropium and procaterol
li. Glycopyrronium, ipratropium and fenoterol
lii. Glycopyrronium, ipratropium and bitolterol
liii. Glycopyrronium, ipratropium and ritodrine
liv. Glycopyrronium, ipratropium and metaproterenol
lv. Glycopyrronium, oxitropium and salbutamol
lvi. Glycopyrronium, oxitropium and levosalbutamol
lvii. Glycopyrronium, oxitropium and terbutaline
lviii. Glycopyrronium, oxitropium and pirbuterol
lix. Glycopyrronium, oxitropium and procaterol
lx. Glycopyrronium, oxitropium and fenoterol
lxi. Glycopyrronium, oxitropium and bitolterol
lxii. Glycopyrronium, oxitropium and ritodrine
lxiii. Glycopyrronium, oxitropium and metaproterenol
lxiv. Daratropium, tiotropium and salbutamol
lxv. Daratropium, tiotropium and levosalbutamol
lxvi. Daratropium, tiotropium and terbutaline
lxvii. Daratropium, tiotropium and pirbuterol
lxviii. Daratropium, tiotropium and procaterol
lxix. Daratropium, tiotropium and fenoterol
lxx. Daratropium, tiotropium and bitolterol
lxxi. Daratropium, tiotropium and ritodrine
lxxii. Daratropium, tiotropium and metaproterenol
lxxiii. Daratropium, aclidinium and salbutamol
lxxiv. Daratropium, aclidinium and levosalbutamol
lxxv. Daratropium, aclidinium and terbutaline
lxxvi. Daratropium, aclidinium and pirbuterol
lxxvii. Daratropium, aclidinium and procaterol
lxxviii. Daratropium, aclidinium and fenoterol
lxxix. Daratropium, aclidinium and bitolterol
lxxx. Daratropium, aclidinium and ritodrine
lxxxi. Daratropium, aclidinium and metaproterenol
lxxxii. Daratropium, glycopyrronium and salbutamol
lxxxiii. Daratropium, glycopyrronium and levosalbutamol
lxxxiv. Daratropium, glycopyrronium and terbutaline
lxxxv. Daratropium, glycopyrronium and pirbuterol
lxxxvi. Daratropium, glycopyrronium and procaterol
lxxxvii. Daratropium, glycopyrronium and fenoterol
lxxxviii. Daratropium, glycopyrronium and bitolterol
lxxxix. Daratropium, glycopyrronium and ritodrine
xc. Daratropium, glycopyrronium and metaproterenol
xci. Daratropium, ipratropium and salbutamol
xcii. Daratropium, ipratropium and levosalbutamol
xciii. Daratropium, ipratropium and terbutaline
xciv. Daratropium, ipratropium and pirbuterol
xcv. Daratropium, ipratropium and procaterol
xcvi. Daratropium, ipratropium and fenoterol
xcvii. Daratropium, ipratropium and bitolterol
xcviii. Daratropium, ipratropium and ritodrine
xcix. Daratropium, ipratropium and metaproterenol
c. Daratropium, oxitropium and salbutamol
ci. Daratropium, oxitropium and levosalbutamol
cii. Daratropium, oxitropium and terbutaline
ciii. Daratropium, oxitropium and pirbuterol
civ. Daratropium, oxitropium and procaterol
cv. Daratropium, oxitropium and fenoterol
cvi. Daratropium, oxitropium and bitolterol
cvii. Daratropium, oxitropium and ritodrine
cviii. Daratropium, oxitropium and metaproterenol
cix. Indacaterol, tirotropium and salbutamol
cx. Indacaterol, tirotropium and levosalbutamol
cxi. Indacaterol, tirotropium and terbutaline
cxii. Indacaterol, tirotropium and pirbuterol
cxiii. Indacaterol, tirotropium and procaterol
cxiv. Indacaterol, tirotropium and fenoterol
cxv. Indacaterol, tirotropium and bitolterol
cxvi. Indacaterol, tirotropium and ritodrine
cxvii. Indacaterol, tirotropium and metaproterenol
cxviii. Indacaterol, glycopyrronium and salbutamol
cxix. Indacaterol, glycopyrronium and levosalbutamol
cxx. Indacaterol, glycopyrronium and terbutaline
cxxi. Indacaterol, glycopyrronium and pirbuterol
cxxii. Indacaterol, glycopyrronium and procaterol
cxxiii. Indacaterol, glycopyrronium and fenoterol
cxxiv. Indacaterol, glycopyrronium and bitolterol
cxxv. Indacaterol, glycopyrronium and ritodrine
cxxvi. Indacaterol, glycopyrronium and metaproterenol
cxxvii. Indacaterol, aclidinium and salbutamol
cxxviii. Indacaterol, aclidinium and levosalbutamol
cxxix. Indacaterol, aclidinium and terbutaline
cxxx. Indacaterol, aclidinium and pirbuterol
cxxxi. Indacaterol, aclidinium and procaterol
cxxxii. Indacaterol, aclidinium and fenoterol
cxxxiii. Indacaterol, aclidinium and bitolterol
cxxxiv. Indacaterol, aclidinium and ritodrine
cxxxv. Indacaterol, aclidinium and metaproterenol
cxxxvi. Indacaterol, ipratropium and salbutamol
cxxxvii. Indacaterol, ipratropium and levosalbutamol
cxxxviii. Indacaterol, ipratropium and terbutaline
cxxxix. Indacaterol, ipratropium and pirbuterol
cxl. Indacaterol, ipratropium and procaterol
cxli. Indacaterol, ipratropium and fenoterol
cxlii. Indacaterol, ipratropium and bitolterol
cxliii. Indacaterol, ipratropium and ritodrine
cxliv. Indacaterol, ipratropium and metaproterenol
cxlv. Indacaterol, oxitropium and salbutamol
cxlvi. Indacaterol, oxitropium and levosalbutamol
cxlvii. Indacaterol, oxitropium and terbutaline
cxlviii. Indacaterol, oxitropium and pirbuterol
cxlix. Indacaterol, oxitropium and procaterol
cl. Indacaterol, oxitropium and fenoterol
cli. Indacaterol, oxitropium and bitolterol
clii. Indacaterol, oxitropium and ritodrine
cliii. Indacaterol, oxitropium and metaproterenol
cliv. Vilanterol, tiotropium and salbutamol
clv. Vilanterol, tiotropium and levosalbutamol
clvi. Vilanterol, tiotropium and terbutaline
clvii. Vilanterol, tiotropium and pirbuterol
clviii. Vilanterol, tiotropium and procaterol
clix. Vilanterol, tiotropium and fenoterol
clx. Vilanterol, tiotropium and bitolterol
clxi. Vilanterol, tiotropium and ritodrine
clxii. Vilanterol, tiotropium and metaproterenol
clxiii. Vilanterol, glycopyrronium and salbutamol
clxiv. Vilanterol, glycopyrronium and levosalbutamol
clxv. Vilanterol, glycopyrronium and terbutaline
clxvi. Vilanterol, glycopyrronium and pirbuterol
clxvii. Vilanterol, glycopyrronium and procaterol
clxviii. Vilanterol, glycopyrronium and fenoterol
clxix. Vilanterol, glycopyrronium and bitolterol
clxx. Vilanterol, glycopyrronium and ritodrine
clxxi. Vilanterol, glycopyrronium and metaproterenol
clxxii. Vilanterol, ipratropium and salbutamol
clxxiii. Vilanterol, ipratropium and levosalbutamol
clxxiv. Vilanterol, ipratropium and terbutaline
clxxv. Vilanterol, ipratropium and pirbuterol
clxxvi. Vilanterol, ipratropium and procaterol
clxxvii. Vilanterol, ipratropium and fenoterol
clxxviii. Vilanterol, ipratropium and bitolterol
clxxix. Vilanterol, ipratropium and ritodrine
clxxx. Vilanterol, ipratropium and metaproterenol
clxxxi. Vilanterol, aclidinium and salbutamol
clxxxii. Vilanterol, aclidinium and levosalbutamol
clxxxiii. Vilanterol, aclidinium and terbutaline
clxxxiv. Vilanterol, aclidinium and pirbuterol
clxxxv. Vilanterol, aclidinium and procaterol
clxxxvi. Vilanterol, aclidinium and fenoterol
clxxxvii. Vilanterol, aclidinium and bitolterol
clxxxviii. Vilanterol, aclidinium and ritodrine
clxxxix. Vilanterol, aclidinium and metaproterenol
cxc. Vilanterol, oxitropium and salbutamol
cxci. Vilanterol, oxitropium and levosalbutamol
cxcii. Vilanterol, oxitropium and terbutaline
cxciii. Vilanterol, oxitropium and pirbuterol
cxciv. Vilanterol, oxitropium and procaterol
cxcv. Vilanterol, oxitropium and fenoterol
cxcvi. Vilanterol, oxitropium and bitolterol
cxcvii. Vilanterol, oxitropium and ritodrine
cxcviii. Vilanterol, oxitropium and metaproterenol
cxcix. Carmoterol, tiotropium and salbutamol
cc. Carmoterol, tiotropium and levosalbutamol
cci. Carmoterol, tiotropium and terbutaline
ccii. Carmoterol, tiotropium and pirbuterol
cciii. Carmoterol, tiotropium and procaterol
cciv. Carmoterol, tiotropium and fenoterol
ccv. Carmoterol, tiotropium and bitolterol
ccvi. Carmoterol, tiotropium and ritodrine
ccvii. Carmoterol, tiotropium and metaproterenol
ccviii. Carmoterol, ipratropium and levosalbutamol
ccix. Carmoterol, ipratropium and salbutamol
ccx. Carmoterol, ipratropium and terbutaline
ccxi. Carmoterol, ipratropium and pirbuterol
ccxii. Carmoterol, ipratropium and procaterol
ccxiii. Carmoterol, ipratropium and fenoterol
ccxiv. Carmoterol, ipratropium and bitolterol
ccxv. Carmoterol, ipratropium and ritodrine
ccxvi. Carmoterol, ipratropium and metaproterenol
ccxvii. Carmoterol, aclidinum and levosalbutamol
ccxviii. Carmoterol, aclidinum and salbutamol
ccxix. Carmoterol, aclidinum and terbutaline
ccxx. Carmoterol, aclidinum and pirbuterol
ccxxi. Carmoterol, aclidinum and procaterol
ccxxii. Carmoterol, aclidinum and fenoterol
ccxxiii. Carmoterol, aclidinum and bitolterol
ccxxiv. Carmoterol, aclidinum and ritodrine
ccxxv. Carmoterol, aclidinum and metaproterenol
ccxxvi. Carmoterol, oxitropium and salbutamol
ccxxvii. Carmoterol, oxitropium and levosalbutamol
ccxxviii. Carmoterol, oxitropium and terbutaline
ccxxix. Carmoterol, oxitropium and pirbuterol
ccxxx. Carmoterol, oxitropium and procaterol
ccxxxi. Carmoterol, oxitropium and fenoterol
ccxxxii. Carmoterol, oxitropium and bitolterol
ccxxxiii. Carmoterol, oxitropium and ritodrine
ccxxxiv. Carmoterol, oxitropium and metaproterenol
ccxxxv. Olodaterol, tiotropium and salbutamol
ccxxxvi. Olodaterol, tiotropium and levosalbutamol
ccxxxvii. Olodaterol, tiotropium and terbutaline
ccxxxviii. Olodaterol, tiotropium and pirbuterol
ccxxxix. Olodaterol, tiotropium and procaterol
ccxl. Olodaterol, tiotropium and fenoterol
ccxli. Olodaterol, tiotropium and bitolterol
ccxlii. Olodaterol, tiotropium and ritodrine
ccxliii. Olodaterol, tiotropium and metaproterenol
ccxliv. Olodaterol, ipratropium and salbutamol
ccxlv. Olodaterol, ipratropium and levosalbutamol
ccxlvi. Olodaterol, ipratropium and terbutaline
ccxlvii. Olodaterol, ipratropium and pirbuterol
ccxlviii. Olodaterol, ipratropium and procaterol
ccxlix. Olodaterol, ipratropium and fenoterol
ccl. Olodaterol, ipratropium and bitolterol
ccli. Olodaterol, ipratropium and ritodrine
cclii. Olodaterol, ipratropium and metaproterenol
ccliii. Olodaterol, aclidinum and salbutamol
ccliv. Olodaterol, aclidinum and levosalbutamol
cclv. Olodaterol, aclidinum and terbutaline
cclvi. Olodaterol, aclidinum and pirbuterol
cclvii. Olodaterol, aclidinum and procaterol
cclviii. Olodaterol, aclidinum and fenoterol
cclix. Olodaterol, aclidinum and bitolterol
cclx. Olodaterol, aclidinum and ritodrine
cclxi. Olodaterol, aclidinum and metaproterenol
cclxii. Olodaterol, glycopyrronium and salbutamol
cclxiii. Olodaterol, glycopyrronium and levosalbutamol
cclxiv. Olodaterol, glycopyrronium and terbutaline
cclxv. Olodaterol, glycopyrronium and pirbuterol
cclxvi. Olodaterol, glycopyrronium and procaterol
cclxvii. Olodaterol, glycopyrronium and fenoterol
cclxviii. Olodaterol, glycopyrronium and bitolterol
cclxix. Olodaterol, glycopyrronium and ritodrine
cclxx. Olodaterol, glycopyrronium and metaproterenol
cclxxi. Olodaterol, oxitropium and salbutamol
cclxxii. Olodaterol, oxitropium and levosalbutamol
cclxxiii. Olodaterol, oxitropium and terbutaline
cclxxiv. Olodaterol, oxitropium and pirbuterol
cclxxv. Olodaterol, oxitropium and procaterol
cclxxvi. Olodaterol, oxitropium and fenoterol
cclxxvii. Olodaterol, oxitropium and bitolterol
cclxxviii. Olodaterol, oxitropium and ritodrine
cclxxix. Olodaterol, oxitropium and metaproterenol
wherein the above therapeutic agents can be present as a pharmaceutically acceptable salt or ester thereof, or in enantiomerically pure form or as a racemic mixture.

42. The pharmaceutical composition according to claims 28, comprising any of the following combinations, said combinations being suitable for administration separately, sequentially or together in effective amounts;
i. Aclidinum, tiotropium and fluticasone
ii. Aclidinum, tiotropium and ciclesonide
iii. Aclidinum, tiotropium and budesonide
iv. Aclidinum, tiotropium and mometasone
v. Aclidinum, tiotropium and beclomethasone
vi. Aclidinum, tiotropium and triamcinolone
vii. Aclidinum, tiotropium and flunisolide
viii. Aclidinum, tiotropium and dexomethasone
ix. Daratropium, tiotropium and fluticasone
x. Daratropium, tiotropium and ciclesonide
xi. Daratropium, tiotropium and budesonide
xii. Daratropium, tiotropium and mometasone
xiii. Daratropium, tiotropium and beclamethasone
xiv. Daratropium, tiotropium and triamcinolone
xv. Daratropium, tiotropium and flunisolide
xvi. Daratropium, tiotropium and dexomethasone
xvii. Indacaterol, tiotropium and fluticasone
xviii. Indacaterol, tiotropium and budesonide
xix. Indacaterol, tiotropium and ciclesonide
xx. Indacaterol, tiotropium and mometasone
xxi. Indacaterol, tiotropium and beclamethasone
xxii. Indacaterol, tiotropium and triamcinolone
xxiii. Indacaterol, tiotropium and flunisolide
xxiv. Indacaterol, tiotropium and dexomethasone
xxv. Vilanterol, tiotropium and ciclesonide
xxvi. vilanterol, tiotropium and fluticasone
xxvii. vilanterol, tiotropium and budesonide
xxviii. vilanterol, tiotropium and mometasone
xxix. vilanterol, tiotropium and beclamethasone
xxx. vilanterol, tiotropium and triamcinolone
xxxi. vilanterol, tiotropium and flunisolide
xxxii. vilanterol, tiotropium and dexomethasone
xxxiii. carmoterol, tiotropium and budesonide
xxxiv. carmoterol, tiotropium and ciclesonide
xxxv. carmoterol, tiotropium and fluticasone
xxxvi. carmoterol, tiotropium and mometasone
xxxvii. carmoterol, tiotropium and beclamethasone
xxxviii. carmoterol, tiotropium and triamcinolone
xxxix. carmoterol, tiotropium and flunisolide
xl. carmoterol, tiotropium and dexomethasone
xli. Olodaterol, tiotropium and ciclesonide
xlii. Olodaterol, tiotropium and fluticasone
xliii. Olodaterol, tiotropium and budesonide
xliv. Olodaterol, tiotropium and mometasone
xlv. Olodaterol, tiotropium and beclamethasone
xlvi. Olodaterol, tiotropium and triamcinolone
xlvii. Olodaterol, tiotropium and flunisolide
xlviii. Olodaterol, tiotropium and dexomethasone
wherein the above therapeutic agents can be present as a pharmaceutically acceptable salt or ester thereof, or in enantiomerically pure form or as a racemic mixture.

43. The pharmaceutical composition according to claims 29, comprising any of the following combinations, said combinations being suitable for administration separately, sequentially or together in effective amounts;
i. Aclidinium, salmeterol and salbutamol
ii. Aclidinium, salmeterol and levosalbutamol
iii. Aclidinium, formoterol and salbutamol
iv. Aclidinium, formoterol and levosalbutamol
v. Aclidinium, arformoterol and salbutamol
vi. Aclidinium, arformoterol and levosalbutamol
vii. Aclidinium, indacaterol and salbutamol
viii. Aclidinium, indacaterol and levosalbutamol
ix. Aclidinium, olodaterol and salbutamol
x. Aclidinium, olodaterol and levosalbutamol
xi. Aclidinium, vilanterol and salbutamol
xii. Aclidinium, vilanterol and levosalbutamol
xiii. Aclidinium, carmoterol and salbutamol
xiv. Aclidinium, carmoterol and levosalbutamol
xv. Aclidinium, bambuterol and salbutamol
xvi. Aclidinium, bambuterol and levosalbutamol
xvii. Glycopyrronium, indacaterol and salbutamol
xviii. Glycopyrronium, indacaterol and levosalbutamol
xix. Glycopyrronium, salmeterol and salbutamol
xx. Glycopyrronium, salmeterol and levosalbutamol
xxi. Glycopyrronium, formoterol and salbutamol
xxii. Glycopyrronium, formoterol and levosalbutamol
xxiii. Glycopyrronium, arformoterol and salbutamol
xxiv. Glycopyrronium, arformoterol and levosalbutamol
xxv. Glycopyrronium, carmoterol and salbutamol
xxvi. Glycopyrronium, carmoterol and levosalbutamol
xxvii. Glycopyrronium, olodaterol and salbutamol
xxviii. Glycopyrronium, olodaterol and levosalbutamol
xxix. Glycopyrronium, vilanterol and salbutamol
xxx. Glycopyrronium, vilanterol and levosalbutamol
xxxi. Glycopyrronium, bambuterol and salbutamol
xxxii. Glycopyrronium, bambuterol and levosalbutamol
xxxiii. Daratropium, indacaterol and salbutamol
xxxiv. Daratropium, indacaterol and levosalbutamol
xxxv. Daratropium, salmeterol and salbutamol
xxxvi. Daratropium, salmeterol and levosalbutamol
xxxvii. Daratropium, formoterol and salbutamol
xxxviii. Daratropium, formoterol and levosalbutamol
xxxix. Daratropium, carmoterol and salbutamol
xl. Daratropium, carmoterol and levosalbutamol
xli. Daratropium, olodaterol and salbutamol
xlii. Daratropium, olodaterol and levosalbutamol
xliii. Daratropium, vilanterol and salbutamol
xliv. Daratropium, vilanterol and levosalbutamol
xlv. Daratropium, bambuterol and salbutamol
xlvi. Daratropium, bambuterol and levosalbutamol
xlvii. Daratropium, arformoterol and salbutamol
xlviii. Daratropium, arformoterol and levosalbutamol
wherein the above therapeutic agents can be present as a pharmaceutically acceptable salt or ester thereof, or in enantiomerically pure form or as a racemic mixture.

44. The pharmaceutical composition according to claims 30, comprising any of the following combinations, said combinations being suitable for administration separately, sequentially or together in effective amounts;
i. Aclidinium, salmeterol and mometasone
ii. Aclidinium, salmeterol and fluticasone
iii. Aclidinium, salmeterol and budesonide
iv. Aclidinium, formoterol and mometasone
v. Aclidinium, formoterol and fluticasone
vi. Aclidinium, formoterol and budesonide
vii. Aclidinium, arformoterol and mometasone
viii. Aclidinium, arformoterol and fluticasone
ix. Aclidinium, arformoterol and budesonide
x. Aclidinium, indacaterol and mometasone
xi. Aclidinium, indacaterol and fluticasone
xii. Aclidinium, indacaterol and budesonide
xiii. Aclidinium, olodaterol and mometasone
xiv. Aclidinium, olodaterol and fluticasone
xv. Aclidinium, olodaterol and budesonide
xvi. Aclidinium, vilanterol and mometasone
xvii. Aclidinium, vilanterol and fluticasone
xviii. Aclidinium, vilanterol and budesonide
xix. Aclidinium, carmoterol and mometasone
xx. Aclidinium, carmoterol and fluticasone
xxi. Aclidinium, carmoterol and budesonide
xxii. Aclidinium, bambuterol and mometasone
xxiii. Aclidinium, bambuterol and fluticasone
xxiv. Aclidinium, bambuterol and budesonide
xxv. Glycopyrronium, indacaterol and mometasone
xxvi. Glycopyrronium, indacaterol and fluticasone
xxvii. Glycopyrronium, indacaterol and budesonide
xxviii. Glycopyrronium, salmeterol and mometasone
xxix. Glycopyrronium, salmeterol and fluticasone
xxx. Glycopyrronium, salmeterol and budesonide
xxxi. Glycopyrronium, formoterol and mometasone
xxxii. Glycopyrronium, formoterol and fluticasone
xxxiii. Glycopyrronium, formoterol and budesonide
xxxiv. Glycopyrronium, arformoterol and mometasone
xxxv. Glycopyrronium, arformoterol and fluticasone
xxxvi. Glycopyrronium, arformoterol and budesonide
xxxvii. Glycopyrronium, carmoterol and mometasone
xxxviii. Glycopyrronium, carmoterol and fluticasone
xxxix. Glycopyrronium, carmoterol and budesonide
xl. Glycopyrronium, olodaterol and mometasone
xli. Glycopyrronium, olodaterol and fluticasone
xlii. Glycopyrronium, olodaterol and budesonide
xliii. Glycopyrronium, vilanterol and mometasone
xliv. Glycopyrronium, vilanterol and fluticasone
xlv. Glycopyrronium, vilanterol and budesonide
xlvi. Glycopyrronium, bambuterol and mometasone
xlvii. Glycopyrronium, bambuterol and fluticasone
xlviii. Glycopyrronium, bambuterol and budesonide
xlix. Daratropium, indacaterol and mometasone
l. Daratropium, indacaterol and fluticasone
li. Daratropium, indacaterol and budesonide
lii. Daratropium, salmeterol and mometasone
liii. Daratropium, salmeterol and fluticasone
liv. Daratropium, salmeterol and budesonide
lv. Daratropium, formoterol and mometasone
lvi. Daratropium, formoterol and fluticasone
lvii. Daratropium, formoterol and budesonide
lviii. Daratropium, carmoterol and mometasone
lix. Daratropium, carmoterol and fluticasone
lx. Daratropium, carmoterol and budesonide
lxi. Daratropium, olodaterol and mometasone
lxii. Daratropium, olodaterol and fluticasone
lxiii. Daratropium, olodaterol and budesonide
lxiv. Daratropium, vilanterol and mometasone
lxv. Daratropium, vilanterol and fluticasone
lxvi. Daratropium, vilanterol and budesonide
lxvii. Daratropium, bambuterol and mometasone
lxviii. Daratropium, bambuterol and fluticasone
lxix. Daratropium, bambuterol and budesonide
lxx. Daratropium, arformoterol and mometasone
lxxi. Daratropium, arformoterol and fluticasone
lxxii. Daratropium, arformoterol and budesonide
lxxiii. Indacaterol, salmeterol and mometasone
lxxiv. Indacaterol, salmeterol and fluticasone
lxxv. Indacaterol, salmeterol and budesonide
lxxvi. Indacaterol, formoterol and mometasone
lxxvii. Indacaterol, formoterol and fluticasone
lxxviii. Indacaterol, formoterol and budesonide
lxxix. Indacaterol, arformoterol and mometasone
lxxx. Indacaterol, arformoterol and fluticasone
lxxxi. Indacaterol, arformoterol and budesonide
lxxxii. Indacaterol, olodaterol and mometasone
lxxxiii. Indacaterol, olodaterol and fluticasone
lxxxiv. Indacaterol, olodaterol and budesonide
lxxxv. Indacaterol, vilanterol and mometasone
lxxxvi. Indacaterol, vilanterol and fluticasone
lxxxvii. Indacaterol, vilanterol and budesonide
lxxxviii. Indacaterol, carmeterol and mometasone
lxxxix. Indacaterol, carmeterol and fluticasone
xc. Indacaterol, carmeterol and budesonide
xci. Indacaterol, bambuterol and mometasone
xcii. Indacaterol, bambuterol and fluticasone
xciii. Indacaterol, bambuterol and budesonide
xciv. Vilanterol, salmeterol and mometasone
xcv. Vilanterol, salmeterol and fluticasone
xcvi. Vilanterol, salmeterol and budesonide
xcvii. Vilanterol, formoterol and mometasone
xcviii. Vilanterol, formoterol and fluticasone
xcix. Vilanterol, formoterol and budesonide
c. Vilanterol, arformoterol and mometasone
ci. Vilanterol, arformoterol and fluticasone
cii. Vilanterol, arformoterol and budesonide
ciii. Vilanterol, olodaterol and mometasone
civ. Vilanterol, olodaterol and fluticasone
cv. Vilanterol, olodaterol and budesonide
cvi. Vilanterol, carmeterol and mometasone
cvii. Vilanterol, carmeterol and fluticasone
cviii. Vilanterol, carmeterol and budesonide
cix. Vilanterol, bambuterol and mometasone
cx. Vilanterol, bambuterol and fluticasone
cxi. Vilanterol, bambuterol and budesonide
cxii. Vilanterol, indacaterol and mometasone
cxiii. Vilanterol, indacaterol and fluticasone
cxiv. Vilanterol, indacaterol and budesonide
cxv. Carmeterol, salmeterol and mometasone
cxvi. Carmeterol, salmeterol and fluticasone
cxvii. Carmeterol, salmeterol and budesonide
cxviii. Carmeterol, formoterol and mometasone
cxix. Carmeterol, formoterol and fluticasone
cxx. Carmeterol, formoterol and budesonide
cxxi. Carmeterol, arformoterol and mometasone
cxxii. Carmeterol, arformoterol and fluticasone
cxxiii. Carmeterol, arformoterol and budesonide
cxxiv. Carmeterol, indaceterol and mometasone
cxxv. Carmeterol, indaceterol and fluticasone
cxxvi. Carmeterol, indaceterol and budesonide
cxxvii. Carmeterol, olodaterol and mometasone
cxxviii. Carmeterol, olodaterol and fluticasone
cxxix. Carmeterol, olodaterol and budesonide
cxxx. Carmeterol, vilanterol and mometasone
cxxxi. Carmeterol, vilanterol and fluticasone
cxxxii. Carmeterol, vilanterol and budesonide
cxxxiii. Carmeterol, bambuterol and mometasone
cxxxiv. Carmeterol, bambuterol and fluticasone
cxxxv. Carmeterol, bambuterol and budesonide
cxxxvi. Olodaterol, salmeterol and mometasone
cxxxvii. Olodaterol, salmeterol and fluticasone
cxxxviii. Olodaterol, salmeterol and budesonide
cxxxix. Olodaterol, formoterol and mometasone
cxl. Olodaterol, formoterol and fluticasone
cxli. Olodaterol, formoterol and budesonide
cxlii. Olodaterol, arformoterol and mometasone
cxliii. Olodaterol, arformoterol and fluticasone
cxliv. Olodaterol, arformoterol and budesonide
cxlv. Olodaterol, indacaterol and mometasone
cxlvi. Olodaterol, indacaterol and fluticasone
cxlvii. Olodaterol, indacaterol and budesonide
cxlviii. Olodaterol, vilanterol and mometasone
cxlix. Olodaterol, vilanterol and fluticasone
cl. Olodaterol, vilanterol and budesonide
cli. Olodaterol, carmeterol and mometasone
clii. Olodaterol, carmeterol and fluticasone
cliii. Olodaterol, carmeterol and budesonide
cliv. Olodaterol, bambuterol and mometasone
clv. Olodaterol, bambuterol and fluticasone
clvi. Olodaterol, bambuterol and budesonide
wherein the above therapeutic agents can be present as a pharmaceutically acceptable salt or ester thereof, or in enantiomerically pure form or as a racemic mixture.

45. The pharmaceutical composition according to claims 31, comprising any of the following combinations, said combinations being suitable for administration separately, sequentially or together in effective amounts;
i. Aclidinium, salbutamol and fluticasone
ii. Aclidinium, levosalbutamol and fluticasone
iii. Aclidinium, salbutamol and ciclesonide
iv. Aclidinium, levosalbutamol and ciclesonide
v. Aclidinium, salbutamol and budesonide
vi. Aclidinium, levosalbutamol and budesonide
vii. Aclidinium, salbutamol and mometasone
viii. Aclidinium, levosalbutamol and mometasone
ix. Aclidinium, salbutamol and beclometahsone
x. Aclidinium, levosalbutamol and beclometahsone
xi. Aclidinium, salbutamol and triamcinolone
xii. Aclidinium, levosalbutamol and triamcinolone
xiii. Aclidinium, salbutamol and flunisolide
xiv. Aclidinium, levosalbutamol and flunisolide
xv. Aclidinium, salbutamol and dexamethasone
xvi. Aclidinium, levosalbutamol and dexamethasone
xvii. Glycopyrronium, salbutamol and fluticasone
xviii. Glycopyrronium, levosalbutamol and fluticasone
xix. Glycopyrronium, salbutamol and ciclesonide
xx. Glycopyrronium, levosalbutamol and ciclesonide
xxi. Glycopyrronium, salbutamol and budesonide
xxii. Glycopyrronium, levosalbutamol and budesonide
xxiii. Glycopyrronium, salbutamol and mometasone
xxiv. Glycopyrronium, levosalbutamol and mometasone
xxv. Glycopyrronium, salbutamol and beclometahsone
xxvi. Glycopyrronium, levosalbutamol and beclometahsone
xxvii. Glycopyrronium, salbutamol and triamcinolone
xxviii. Glycopyrronium, levosalbutamol and triamcinolone
xxix. Glycopyrronium, salbutamol and flunisolide
xxx. Glycopyrronium, levosalbutamol and flunisolide
xxxi. Glycopyrronium, salbutamol and dexamethasone
xxxii. Glycopyrronium, levosalbutamol and dexamethasone
xxxiii. Daratropium, salbutamol and fluticasone
xxxiv. Daratropium, levosalbutamol and fluticasone
xxxv. Daratropium, salbutamol and ciclesonide
xxxvi. Daratropium, levosalbutamol and ciclesonide
xxxvii. Daratropium, salbutamol and budesonide
xxxviii. Daratropium, levosalbutamol and budesonide
xxxix. Daratropium, salbutamol and mometasone
xl. Daratropium, levosalbutamol and mometasone
xli. Daratropium, salbutamol and beclometahsone
xlii. Daratropium, levosalbutamol and beclometahsone
xliii. Daratropium, salbutamol and triamcinolone
xliv. Daratropium, levosalbutamol and triamcinolone
xlv. Daratropium, salbutamol and flunisolide
xlvi. Daratropium, levosalbutamol and flunisolide
xlvii. Daratropium, salbutamol and dexamethasone
xlviii. Daratropium, levosalbutamol and dexamethasone
xlix. Indacaterol, salbutamol and fluticasone
l. Indacaterol, levosalbutamol and fluticasone
li. Indacaterol, salbutamol and ciclesonide
lii. Indacaterol, levosalbutamol and ciclesonide
liii. Indacaterol, salbutamol and budesonide
liv. Indacaterol, levosalbutamol and budesonide
lv. Indacaterol, salbutamol and mometasone
lvi. Indacaterol, levosalbutamol and mometasone
lvii. Indacaterol, salbutamol and beclometahsone
lviii. Indacaterol, levosalbutamol and beclometahsone
lix. Indacaterol, salbutamol and triamcinolone
lx. Indacaterol, levosalbutamol and triamcinolone
lxi. Indacaterol, salbutamol and flunisolide
lxii. Indacaterol, levosalbutamol and flunisolide
lxiii. Indacaterol, salbutamol and dexamethasone
lxiv. Indacaterol, levosalbutamol and dexamethasone
lxv. Vilanterol, salbutamol and fluticasone
lxvi. Vilanterol, levosalbutamol and fluticasone
lxvii. Vilanterol, salbutamol and budesonide
lxviii. Vilanterol, levosalbutamol and budesonide
lxix. Vilanterol, salbutamol and ciclesonide
lxx. Vilanterol, levosalbutamol and ciclesonide
lxxi. Vilanterol, salbutamol and mometasone
lxxii. Vilanterol, levosalbutamol and mometasone
lxxiii. Vilanterol, salbutamol and beclomethasone
lxxiv. Vilanterol, levosalbutamol and beclomethasone
lxxv. Vilanterol, salbutamol and triamcinolone
lxxvi. Vilanterol, levosalbutamol and triamcinolone
lxxvii. Vilanterol, salbutamol and flunisolide
lxxviii. Vilanterol, levosalbutamol and flunisolide
lxxix. Vilanterol, salbutamol and dexamethasone
lxxx. Vilanterol, levosalbutamol and dexamethasone
lxxxi. Carmeterol, salbutamol and fluticasone
lxxxii. Carmeterol, levosalbutamol and fluticasone
lxxxiii. Carmeterol, salbutamol and ciclesonide
lxxxiv. Carmeterol, levosalbutamol and ciclesonide
lxxxv. Carmeterol, salbutamol and budesonide
lxxxvi. Carmeterol, levosalbutamol and budesonide
lxxxvii. Carmeterol, salbutamol and mometasone
lxxxviii. Carmeterol, levosalbutamol and mometasone
lxxxix. Carmeterol, salbutamol and beclomethasone
xc. Carmeterol, levosalbutamol and beclomethasone
xci. Carmeterol, salbutamol and triamcinolone
xcii. Carmeterol, levosalbutamol and triamcinolone
xciii. Carmeterol, salbutamol and flunisolide
xciv. Carmeterol, levosalbutamol and flunisolide
xcv. Carmeterol, salbutamol and dexamethasone
xcvi. Carmeterol, levosalbutamol and dexamethasone
xcvii. Olodaterol, salbutamol and fluticasone
xcviii. Olodaterol, levosalbutamol and fluticasone
xcix. Olodaterol, salbutamol and ciclesonide
c. Olodaterol, levosalbutamol and ciclesonide
ci. Olodaterol, salbutamol and budesonide
cii. Olodaterol, levosalbutamol and budesonide
ciii. Olodaterol, salbutamol and mometasone
civ. Olodaterol, levosalbutamol and mometasone
cv. Olodaterol, salbutamol and beclomethasone
cvi. Olodaterol, levosalbutamol and beclomethasone
cvii. Olodaterol, salbutamol and triamcinolone
cviii. Olodaterol, levosalbutamol and triamcinolone
cix. Olodaterol, salbutamol and flunisolide
cx. Olodaterol, levosalbutamol and flunisolide
cxi. Olodaterol, salbutamol and dexamethasone
cxii. Olodaterol, levosalbutamol and dexamethasone
wherein the above therapeutic agents can be present as a pharmaceutically acceptable salt or ester thereof, or in enantiomerically pure form or as a racemic mixture.

46. The pharmaceutical composition according to claim 26, comprising any of the following combinations, said combinations being suitable for administration separately, sequentially or together in effective amounts;
i. Formoterol, budesonide and tiotropium
ii. Salmeterol, fluticasone and tiotropium
iii. Carmoterol, tiotropium and fluticasone
iv. Salbutamol, formoterol and budesonide
v. Salbutamol, salmeterol and fluticasone
vi. Salbutamol, arformoterol and fluticasone
wherein the above therapeutic agents can be present as a pharmaceutically acceptable salt or ester thereof, or in enantiomerically pure form or as a racemic mixture.

47. The pharmaceutical composition according to claim 1, wherein the therapeutically effective amount of said pharmaceutical composition is administered once a day.

48. The pharmaceutical composition according to claim 1, wherein the therapeutically effective amount of said pharmaceutical composition is administered twice a day.

49. The pharmaceutical composition according to claim 1, for use in the treatment of respiratory condition selected from asthma and chronic obstructive pulmonary disease and other obstructive airways diseases.

50. The pharmaceutical composition according to claim 1, wherein said pharmaceutical composition being suitable for administration separately, sequentially or together in effective amounts, together with a moisture tight and high barrier sealed blister.

51. The pharmaceutical composition according to claim 1, wherein said pharmaceutical composition being suitable for administration separately, sequentially or together in effective amounts, together with a capsule.

52. The pharmaceutical composition according to claim 50, wherein said pharmaceutical composition being suitable for administration separately, sequentially or together in effective amounts, together with a dry powder inhalation device.

53. The pharmaceutical composition according to claim 50, wherein said pharmaceutical composition being suitable for administration separately, sequentially or together in effective amounts, together with an inhalation device characterized by said device comprise at least one lock mechanism, enabling the device to remain locked in both positions in which the device is ready for inhalation and the lid is in the closed position, and further enabling the device to setup again automatically, when the lid is closed.

54. A pharmaceutical kit comprising the drugs having amine and one or more additional active agents as defined in claim 21, in separate unit dosage forms, said forms being suitable for administration separately, sequentially or together in effective amounts, together with one or more inhalation devices for administration of drugs having amine and one or more additional active agents.
